(19) United States
(12) Patent Application Publication
(10) Pub. No.: US 2016/0015069 A1
(43) Pub. Date: Jan. 21, 2016

(54) KETOGENIC FOOD COMPOSITIONS, METHODS, AND USES THEREOF

(71) Applicant: Pavel Klein, Rockville, MD (US)

(72) Inventor: Pavel KLEIN, Rockville, MD (US)

(73) Assignee: Pavel Klein, Rockville, MD (US)

(21) Appl. No.: 14/798,505

(22) Filed: Jul. 14, 2015

Related U.S. Application Data
(60) Provisional application No. 62/024,827, filed on Jul. 15, 2014.

(51) Int. Cl. A23L 1/29; A61K 38/16; A61K 31/70; A61K 38/17

(52) U.S. Cl. CPC A23L 1/296; A61K 38/1709; A61K 38/168; A61K 38/1706; A61K 31/70; A23L 1/293; A23V 2002/00

(57) ABSTRACT

Disclosed herein is a ketogenic food composition and methods thereof. The ketogenic food composition promotes ketogenesis in a subject in need thereof. The ketogenic food composition includes fat, protein, and carbohydrate. A ratio of fat to a combination of protein and carbohydrate in the ketogenic food composition may be about 2.5:1 to about 4.5:1. The ketogenic food composition may be a sole diet for the subject. Also provided herein is a method of treating a disease in a subject in need thereof. The method includes administering the ketogenic food composition to the subject. The disease may be a cancer or an obesity-related disorder.

### CROSS REFERENCE TO RELATED APPLICATIONS

This application claims priority to U.S. Provisional Application No. 62/024,827 filed, Jul. 15, 2014, the contents of which are incorporated herein by reference in its entirety.

### TECHNICAL FIELD

The present invention relates to a ketogenic food composition, meals, dietary meal plans, methods of manufacturing the same, methods of treatment administering the same, and kits containing the same.

### BACKGROUND

The cells of organisms, including humans, require an energy source to carry out various cellular processes. For many organisms, this cellular energy source is glucose. Glucose is also a primary (or sole) energy source for some cancerous cells and abnormally high levels of glucose can lead to disease such as type 2 diabetes. Another cellular energy source for cells is ketone bodies, which are produced by ketogenesis. Ketone bodies, however, are a less preferred energy source for cells, and thus, are often produced in response to low levels of glucose.

Diets high in fats and low in carbohydrates can result in low glucose levels, thereby promoting ketogenesis, and provide a ready source of fatty acids for ketogenesis. Such diets may be referred to as ketogenic diets. It is difficult, however, to prepare meals for ketogenic diets because such meals are often unpalatable and unvaried. Preparing these meals also requires complicated calculations regarding the overall caloric value of the meal, the calories derived from fat, protein, and carbohydrates for the meal, and the weight of carbohydrates in the meal. Accordingly, individuals often end up preparing meals that are not ketogenic and/or are unable to maintain the ketogenic diet for any length of time.

Accordingly, a need exists in the art for ready-made ketogenic food compositions to facilitate the consumption of a ketogenic diet and treatment of diseases dependent upon glucose metabolism.

Type 2 diabetes is one of the largest U.S. health care cost items. Approximately 90-95% of type 2 diabetes in the U.S. is due to obesity. The risk of developing type 2 diabetes increases in a “dose-dependent” manner with increasing body mass index. See, for example, Colditz G A, Willett W C, Stampfer M J, Manson J E, Hennekens C H, Arky R A, Speizer F E: Weight as a risk factor for clinical diabetes in women. Am J Epidemiol 132:501-513, 1990. Gaining 11 to 16 pounds doubles the risk of Type 2 diabetes; an increase of 17 to 24 pounds nearly triples it. The prevalence of type 2 diabetes is 3-7 times higher in obese than in normal-weight adults, approximately 20 times higher in those with a body mass index, BMI>35 kg/m². See, for example, Colditz, G. A., Willett, W. C., Rotnitzky, A. & Manson, J. E. Weight gain as a risk factor for clinical diabetes mellitus in women. Annals of Internal Medicine, 1995; 122, 7, 481-486 and Field A E, Coakley E H, Must A, Spadano J L, Laird N, Dietz W H, Rimm E, Colditz G A: Impact of overweight on the risk of developing common chronic diseases during a 10-year period. Arch Intern Med 161:1581-1586, 2001.

Rates of type 2 diabetes have increased over the last several decades in parallel with obesity. In 2010 there were approximately 285 million people with the type 2 diabetes worldwide, compared to around 30 million in 1985. See, for example, Williams textbook of endocrinology (12th ed.). Philadelphia: Elsevier/Saunders, 2012. pp. 1371-1435. 34% of US population is obese, defined as body mass index (BMI) of >30 kg/m²; 68% of US adult population is overweight (BMI>25 kg/m²) or obese.

As described herein, the disclosure provides for methods, dietary treatment, meals, and kits for treating a variety of disorders, including type 2 diabetes as well as obesity related type 2 diabetes.

### SUMMARY

The present invention is directed to a ketogenic food composition for promoting ketogenesis in a subject in need thereof. In an aspect, the ketogenic food composition comprises fat, protein, and carbohydrate, wherein a ratio of fat to a combination of protein and carbohydrate is about 2.5:1 to about 4.5:1 (i.e., “the ratio”), and wherein the food composition is a sole diet for the subject.

The ratio may be about 3:1 to about 4:1. The ratio may be about 3:1. The ratio may be about 4:1.

The ketogenic food composition may be a plurality of ketogenic food compositions. In each ketogenic food composition, the ratio may be about 2.5:1 to about 4.5:1. The ratio in each ketogenic food composition may be about 3:1 to about 4:1. The ratio in each ketogenic food composition may be about 3:1. The ratio in each ketogenic food composition may be about 4:1. The plurality of ketogenic food compositions may be three, four, five, fix or more ketogenic food compositions. The ketogenic food compositions may be ingested daily by the subject.

The ketogenic food composition may provide less than or equal to about 20 grams/day of carbohydrates to the subject. The subject may be suffering from a disease selected from the group consisting of: type 2 diabetes, obesity, obstructive sleep apnea, polycystic ovarian syndrome, metabolic syndrome, and any combination thereof. The disease may be type 2 diabetes or obstructive sleep apnea.

The ketogenic food composition may provide less than or equal to about 10 grams/day of carbohydrates to the subject. The subject may be suffering from a cancer. The cancer may be gliobastoma multiforme.

The ketogenic food composition may provide to the subject about 1300 kcal/day to about 2100 kcal/day.

The ketogenic food composition may be frozen, hyalinized, or vacuum-packed or any combination thereof prior to consumption by the subject.

The present invention is also directed to a method for treating a disease in a subject in need thereof. The method comprises administering the above-described ketogenic food composition to the subject. The disease may be a cancer or an obesity-related disorder.

The cancer may be glioblastoma multiforme. The method may further comprise reducing a size of the glioblastoma multiforme relative to a size of the glioblastoma multiforme prior to administration of the above-described ketogenic food composition to the subject. Administration of the above-described ketogenic food composition may be an adjuvant therapy to radiation therapy, chemotherapy, or a combination of radiation therapy and chemotherapy.

The obesity-related disorder may be type 2 diabetes, obstructive sleep apnea, or a combination thereof.

The obesity-related disorder may be type 2 diabetes. The method may further comprise reducing at least one of insulin resistance, blood glucose level, insulin level, obesity, hyperinsulinemia, glycosylated hemoglobin level, or a combination thereof in the subject.

The obesity-related disorder may be obstructive sleep apnea. The method may further comprise reducing the apnea/hyponea index (AHI) in the subject. Reducing the AHI in the subject may include reducing the AHI to normal in the subject.

The ketogenic food composition may be a plurality of ketogenic food compositions. In each ketogenic food composition, the ratio may be about 2.5:1 to about 4.5:1. The ratio in each ketogenic food composition may be about 3:1 to about 4:1. The ratio in each ketogenic food composition may be about 3:1. The ratio in each ketogenic food composition may be about 4:1.

The plurality of ketogenic food compositions may be five ketogenic food compositions. Administering may comprise daily ingestion of the five ketogenic food compositions by the subject. Administering may further comprise daily ingestion for at least about 30 days.

Administering may comprise cyclical ingestion of the plurality of ketogenic food compositions. The cycle may be 5-weeks. The cycle may be 1-month. The cycle may be 2-months. The plurality of ketogenic food compositions may be five ketogenic food compositions ingested per day by the subject.

The present invention is further directed to a kit or meal plan for the treatment of a disease in a subject in need thereof. The kit or meal plan may comprise a plurality of the above-described ketogenic food compositions. The plurality of ketogenic food compositions may be five ketogenic food compositions per day. The disease may be a cancer or an obesity-related disorder. The cancer may be glioblastoma multiforme. The obesity-related disorder may be selected from the group consisting of: type 2 diabetes, obstructive sleep apnea, polycystic ovarian syndrome, and metabolic syndrome.

In an aspect the disclosure provides for a method of treating type 2 diabetes comprising a daily administration to a subject in need thereof of less about 20 g/day of carbohydrates, a fat to a combination of protein and carbohydrate is about 3:1, wherein the daily administration of the meal plan is administered for more than about 2 months, about 4 months, about 6 months or about 12 months or more.

### DETAILED DESCRIPTION

In an aspect, the present invention relates to a ketogenic food composition. The ketogenic food composition promotes ketogenesis in a subject in need thereof. The ketogenic food composition may include fat, protein, and carbohydrate. The ketogenic food composition includes a ratio of fat to a combination of protein and carbohydrate that promotes ketogenesis (i.e., “the ratio”). This ratio may be expressed as a fat concentration to a combination of a protein concentration and a carbohydrate concentration, i.e., [fat]:([protein]+[carbohydrate]). This ratio may about 3:1 or about 4:1. As described in more detail below, multiple ketogenic food compositions (e.g., five ketogenic food compositions) may be ingested by the subject over the course of a day such that a total ratio represents the ratio of fat to a combination of protein and carbohydrate for the entire day. This total ratio may about 3:1 or about 4:1.

In particular, the ketogenic food composition may contain less than or equal to about 20 g carbohydrate per day when the subject is suffering from an obesity-related disorder such as type 2 diabetes, obstructive sleep apnea, polycystic ovarian syndrome, and metabolic syndrome. In an aspect, the daily allowance of 20 g carbohydrate per day does not change throughout the meal plan. The ketogenic food composition may contain less than or equal to about 10 g carbohydrate per day when the subject is suffering from a cancer (e.g., glioblastoma multiforme).

Accordingly, the ketogenic food composition is a ready-made food composition that provides to the subject a predetermined ratio of fat, protein, and carbohydrates. This ratio promotes ketogenesis in a manner to facilitate the treatment of glioblastoma multiforme, type 2 diabetes, obstructive sleep apnea, polycystic ovarian syndrome, and metabolic syndrome. As such, the ketogenic food composition provides ease of preparing a ketogenic diet and meals that are palatable, have good appearance, and provide satiety. This, in turn, allows the subject to easily adhere to the ketogenic diet and thus, improves patient compliance with the treatment, which further leads to unexpected benefits beyond just weight loss.

The present invention also relates to a method of treating a disease in the subject by administering the ketogenic food composition to the subject. This method of treatment significantly reduces the symptoms and/or progression of glioblastoma multiforme, type 2 diabetes, obstructive sleep apnea, polycystic ovarian syndrome, and metabolic syndrome, thereby improving the health the subject. This improvement in health extends beyond the termination of the method of treatment. For example, with regards to type 2 diabetes and glioblastoma multiforme, the health of the subject is improved such that the subject maintains a fasting blood glucose level of less than about 100 mg/dL and a reduction in tumor size, respectively, after termination of the method of treatment. Accordingly, the method of treatment provides health benefits that continue even once the method of treatment is discontinued.

In an aspect, methods, dietary treatments, meals, and kits described herein result in an improvement in type 2 diabetes remission. In an aspect, remission refers to fasting blood glucose or “FBG”<126 mg/dl and a glycated hemoglobin level or “HbA1C”≦6.4% off all diabetic medications at 8 months of treatment. In another aspect, the rate of diabetes type 2 remission is improved relative to individualized programs such as, Look AHEAD (see, for example, Pi-Sunyer X, Blackburn G, Brancati F L, et al.; Look AHEAD Research Group. Reduction in weight and cardiovascular disease risk factors in individuals with type 2 diabetes: one-year results of the Look AHEAD trial. Diabetes Care 2007; 30:1374-1383 and Wing R R, Bolin P, Brancati F L, West D S, Williamson D F, Yanovski S Z. Cardiovascular effects of intensive lifestyle intervention in type 2 diabetes. N Engl J Med. 2013 Jul. 11; 369(2):145-54. doi: 10.1056/NEJMoa1212914. Epub 2013 Jun. 24, which are herein incorporated by reference in their entireties) or WhyWAIT (see, for example, Hamdy O, Carver C. The Why WAIT program: improving clinical outcomes through weight management in type 2 diabetes. Curr Diab Rep. 2008 October; 8(5):413-20, which is herein incorporated by reference in its entirety), which have been shown to result in mild to moderate weight reduction and associated improvement in glycemic control in type 2 diabetes obese patients. However, the rate of Type 2 diabetes remission in these programs is relatively low, and the programs are labor intensive, time consuming and costly, limiting their application on a large scale.

In an aspect, the rate of diabetes type 2 remission for methods, dietary treatments, meals, and kits described herein are improved relative to treatment with a pharmaceutical compound, composition, or medicine capable of treating type 2 diabetes. In an aspect, the rate of diabetes Type 2 remission for methods, dietary treatments, meals, and kits described herein are improved relative to Orlistat (for example, 360 mg/day), Lorcaserin (for example, 20 mg/day), qysimia low, qysimia high, Contrave treatment, Liraglutide (for example, 1.2-1.8 mg/day), or a low carbohydrate diet, such as the Dr. Atkins diet.

In another aspect, subjects treated with methods, dietary treatments, meals, and kits described herein lose about 5% or more, about 10% or more, 15% or more, 20% or more, about 30% or more, or about 50% or more pounds relative to treatment a pharmaceutical compound, composition, or medicine capable of treating type 2 diabetes. In yet another aspect, subjects treated with methods, dietary treatments, meals, and kits described herein lose about 5% or more, about 10% or more, 15% or more, 20% or more, about 30% or more, or about 50% or more pounds relative Orlistat (for example, 360 mg/day), Lorcaserin (for example, 20 mg/day), qysimia low, qysimia high, Contrave treatment, Liraglutide (for example, 1.2-1.8 mg/day), or a low carbohydrate diet, such as the Dr. Atkins, under the same conditions.

In yet another aspect, subjects treated with compositions with methods, dietary treatments, meals, and kits described herein achieve remission of type 2 diabetes in about 1 month, about 2 months, about 3 months, about 4 months, about 6 months, about 8 months, or about 12 months or more. In an aspect, subjects treated with compositions with methods, dietary treatments, meals, and kits described herein do not require prescription and/or consumption of type 2 diabetes pharmaceuticals or medicines after about 2 weeks, 1 month, about 2 months, about 3 months, about 4 months, about 6 months, about 8 months, or about 12 months or more after treatment with compositions with methods, dietary treatments, meals, and kits described herein.

In yet another aspect, subjects diagnosed with Type 2 diabetes treated with methods, dietary treatments, meals, and kits described herein lose about 5% or more, about 10% or more, 15% or more, 20% or more, about 30% or more, or about 50% or more pounds relative to treatment with orlistat (for example, 360 mg/day), Lorcaserin (for example, 20 mg/day), qysimia low, qysimia high, Contrave treatment, Liraglutide (for example, 1.2-1.8 mg/day), or a low carbohydrate diet, such as the Dr. Atkins over about 2 months, about 3 months, about 4 months, about 6 months, or about 12 months or more.

In an aspect, methods, dietary treatments, meals, and kits described herein differ from Atkins diet and other “low carbohydrate” diets in at least two respects: (i) carbohydrate restriction is ≦20 g/day for the duration of the diet, whereas other low carbohydrate diets restrict carbohydrates to 20 g only for the first 2-4 weeks of diet “induction”, after which carbohydrate content is increased up to 40-130 g/day; and, (ii) the fat content is much higher than in low carbohydrate diets: in 3:1 KD 87% of calories are derived from fat vs. 45-50% in low carbohydrate diets which have [fat]:[carbohydrate+protein] ratio of about 1:1.

In an aspect, methods described herein do not require the input or assistance of a chart, meal plan, or instructions that monitor and regulate meal intake. In an aspect, dietitians are not needed to administer a diet of all-containing, pre-figured, ready-made meals described herein. In another aspect, the absolute value of carbohydrates and/or the ratio of fat to carbohydrates and protein does not change during the method or diet plan over the course of administration, for example, about 1 month or more, 2 months or more, about 4 months or more, about 6 months or more, about 12 months or more, or about 24 months or more.

In another aspect, methods, dietary treatments, meals, and kits described herein do not include snacks, such as shakes, bars, and/or liquid supplements.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Apnea/hyponea index” and “AHI” as used herein refer to a measurement of the severity of obstructive sleep apnea. An AHI value reflects the number of apnea or hyponea events per hour of sleep. As the AHI value increases, symptoms of obstructive sleep apnea become more frequent and intense. An AHI value of less than or equal to 5 represents normal. An AHI value of greater than 5 to 14 represents mild obstructive sleep apnea. An AHI value of 15 to 30 represents moderate obstructive sleep apnea. An AHI value of greater than 30 represents severe obstructive sleep apnea.

“Body mass index” and “BMI” as used herein refers to a measurement of body fat based on height and weight. BMI values may have units of kg/m².

“Glioblastoma multiforme” as used herein refers to a cancer or tumor of the central nervous system that forms from glial tissue of the brain and/or spinal cord.

“Obese” as used herein refers to a subject having a body mass index of greater than or equal to 30.0 kg/m².

“Overweight” as used herein refers to a subject having a body mass index of between 25.0 kg/m² and 29.9 kg/m².

“Prediabetic” as used herein refers to a subject having a fasting blood glucose level of from about 100 mg/dL to about 125 mg/dL, a fasting blood glycosylated hemoglobin (HbA1C) level from about 5.7% to about 6.4%, or a combination thereof.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Treat,” “treating,” or “treatment” as used herein interchangeably means to reverse, alleviate, or inhibit the progress of a disease, or one or more symptoms of such disease, to which such term applies. Depending on the condition of the subject, the term also refers to preventing a disease, and includes preventing the onset of a disease, or preventing symptoms associated with a disease. A treatment may be either performed in an acute or chronic way. The term also refers to reducing the severity of a disease or symptoms associated with such disease prior to affliction with the disease. “Preventing” also refers to preventing the recurrence of a disease with one or more symptoms associated with such disease. “Treatment” and “therapeutically” refer to the act of treating as “treating” is defined above.

“Type 2 diabetes” as used herein refers to a subject having a fasting blood glucose level of greater than 126 mg/dL, a fasting blood glycosylated hemoglobin (HbA1C) level greater than 6.4%, or a combination thereof.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 1. KETOGENIC FOOD COMPOSITION

Provided herein is a ketogenic food composition. The ketogenic food composition promotes ketogenesis in a subject in need thereof. The ketogenic food composition increases ketogenesis in the subject relative to ketogenesis in the subject prior to ingestion of the ketogenic food composition.

The ketogenic food composition includes fat, protein, and carbohydrate. The ketogenic food composition includes a ratio of fat to a combination of protein and carbohydrate that promotes ketogenesis. This ratio may be expressed as a fat concentration to a combination of a protein concentration and a carbohydrate concentration, i.e., [fat]:([protein]+[carbohydrate]) (i.e., “the ratio”). As described in more detail below, multiple ketogenic food compositions (e.g., five ketogenic food compositions) may be ingested by the subject over the course of a day such that a total ratio represents the ratio of fat to a combination of protein and carbohydrate for the entire day.

Use of the ketogenic food composition provides other health benefits besides weight loss. Accordingly, the ketogenic food composition is a ready-made food composition that provides to the subject a predetermined ratio of fat, protein, and carbohydrates. This ratio promotes ketogenesis in a manner to facilitate the treatment of an obesity-related disorder (e.g., type 2 diabetes, obstructive sleep apnea, polycystic ovarian syndrome, and metabolic syndrome) and/or a cancer (e.g., glioblastoma multiforme) as described below in more detail. As such, the ketogenic food composition provides ease of preparing a ketogenic diet and meals that are palatable, have good appearance, and provide satiety. This, in turn, allows the subject to easily adhere to the ketogenic diet and thus, improves patient compliance with the treatment.

This ratio of the ketogenic food composition may about 2.5:1 to about 11.0:1, about 2.5:1 to about 10.5:1, about 2.5:1 to about 10.0:1, about 2.5:1 to about 9.5:1, about 2.5:1 to about 9.0:1, about 2.5:1 to about 8.5:1, about 2.5:1 to about 8.0:1, about 2.5:1 to about 7.5:1, about 2.5:1 to about 7.0:1, about 2.5:1 to about 6.5:1, about 2.5:1 to about 6.0:1, about 2.5:1 to about 5.5:1, about 2.5:1 to about 5.0:1, about 2.5:1 to about 4.9:1, about 2.5:1 to about 4.8:1, about 2.5:1 to about 4.7:1, about 2.5:1 to about 4.6:1, about 2.5:1 to about 4.5:1, 2.5:1 to about 4.4:1, about 2.5:1 to about 4.3:1, about 2:5:1 to about 4.2:1, about 2.5:1 to about 4.1:1, about 2.5:1 to about 4.0:1, about 2.6:1 to about 11.0:1, about 2.7:1 to about 11.0:1, about 2.8:1 to about 11.0:1, about 2.9:1 to about 11.0:1, about 3.0:1 to about 11.0:1, about 3.1:1 to about 11.0:1, about 3.2:1 to about 11.0:1, about 3.3:1 to about 11.0:1, about 3.4:1 to about 11.0:1, about 3.5:1 to about 11.0:1, about 3.6:1 to about 11.0:1, about 3.7:1 to about 11.0:1, about 3.8:1 to about 11.0:1 about 3.9:1 to about 11.0:1, about 4.0:1 to about 11.0:1, about 4.1:1 to about 11.0:1, about 4.2:1 to about 11.0:1, about 4.3:1 to about 11.0:1, about 4.4:1 to about 11.0:1, about 4.5:1 to about 11.0:1, about 4.6:1 to about 11.0:1, about 4.7:1 to about 11.0:1, about 4.8:1 to about 11.0:1, about 4.9:1 to about 11.0:1, about 5.0:1 to about 11.0:1, about 5.5:1 to about 11.0:1, about 6.0:1 to about 11.0:1, about 6.5:1 to about 11.0:1, about 7.0:1 to about 11.0:1, about 7.5:1 to about 11.0:1, about 8.0:1 to about 11.0:1, about 8.5:1 to about 11.0:1, about 9.0:1 to about 11.0:1, about 9.5:1 to about 11.0:1, about 10.0:1 to about 11.0:1. The ratio may be about 2.5:1 to about 4.5:1, about 2.6:1 to about 4.5:1, about 2.7:1 to about 4.5:1, about 2.8:1 to about 4.5:1, about 2.9:1 to about 4.5:1, about 3.0 to about 4.5:1, about 2.5:1 to about 4.4:1, about 2.5:1 to about 4.3:1, about 2.5:1 to about 4.2:1, about 2.5:1 to about 4.1:1, about 2.5:1 to about 4.0:1, about 2.6:1 to about 4.4:1, about 2.7:1 to about 4.3:1, about 2.8:1 to about 4.2:1, about 2.9:1 to about 4.1:1, or about 3.0:1 to about 4.0:1. In other embodiments, the ratio may be about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, or about 4.5:1. In still other embodiments, the ratio may be 4.0:1 or 3.0:1.

In some embodiments, the total ratio (i.e., representing the ratio of fat to a combination of protein and carbohydrate for an entire day) may be about 2.5:1 to about 4.5:1, about 2.6:1 to about 4.5:1, about 2.7:1 to about 4.5:1, about 2.8:1 to about 4.5:1, about 2.9:1 to about 4.5:1, about 3.0 to about 4.5:1, about 2.5:1 to about 4.4:1, about 2.5:1 to about 4.3:1, about 2.5:1 to about 4.2:1, about 2.5:1 to about 4.1:1, about 2.5:1 to about 4.0:1, about 2.6:1 to about 4.4:1, about 2.7:1 to about 4.3:1, about 2.8:1 to about 4.2:1, about 2.9:1 to about 4.1:1, or about 3.0:1 to about 4.0:1. In other embodiments, the total ratio may be about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, or about 4.5:1. In other embodiments, this total ratio may be about 4.0:1 or about 3.0:1. In still other embodiments, this total ratio may be 4.0:1 or 3.0:1.

The ketogenic food composition may be a sole source of calories for the subject. The ketogenic food composition may provide about 1300 kilocalories (kcal)/day to about 2100 kcal/day, about 1325 kcal/day to about 2100 kcal/day, about 1350 kcal/day to about 2100 kcal/day, about 1375 kcal/day to about 2100 kcal/day, about 1400 kcal/day to about 2100 kcal/day, about 1425 kcal/day to about 2100 kcal/day, about 1450 kcal/day to about 2100 kcal/day, about 1475 kcal/day to about 2100 kcal/day, about 1500 kcal/day to about 2100 kcal/day, about 1525 kcal/day to about 2100 kcal/day, about 1550 kcal/day to about 2100 kcal/day, about 1575 kcal/day to about 2100 kcal/day, about 1600 kcal/day to about 2100 kcal/day, about 1625 kcal/day to about 2100 kcal/day, about 1650 kcal/day to about 2100 kcal/day, about 1675 kcal/day to about 2100 kcal/day, about 1700 kcal/day to about 2100 kcal/day, about 1725 kcal/day to about 2100 kcal/day, about 1750 kcal/day to about 2100 kcal/day, about 1775 kcal/day to about 2100 kcal/day, about 1800 kcal/day to about 2100 kcal/day, about 1825 kcal/day to about 2100 kcal/day, about 1850 kcal/day to about 2100 kcal/day, about 1875 kcal/day to about 2100 kcal/day, about 1900 kcal/day to about 2100 kcal/day, about 1925 kcal/day to about 2100 kcal/day, about 1950 kcal/day to about 2100 kcal/day, about 1975 kcal/day to about 2100 kcal/day, about 2000 kcal/day to about 2100 kcal/day, about 1300 kcal/day to about 2075 kcal/day, about 1300 kcal/day to about 2050 kcal/day, about 1300 kcal/day to about 2025 kcal/day, about 1300 kcal/day to about 2000 kcal/day, about 1300 kcal/day to about 1975 kcal/day, about 1300 kcal/day to about 1950 kcal/day, about 1300 kcal/day to about 1925 kcal/day, about 1300 kcal/day to about 1900 kcal/day, about 1300 kcal/day to about 1875 kcal/day, about 1300 kcal/day to about 1850 kcal/day, about 1300 kcal/day to about 1825 kcal/day, about 1300 kcal/day to about 1800 kcal/day, about 1300 kcal/day to about 1775 kcal/day, about 1300 kcal/day to about 1750 kcal/day, about 1300 kcal/day to about 1725 kcal/day, about 1300 kcal/day to about 1700 kcal/day, about 1300 kcal/day to about 1675 kcal/day, about 1300 kcal/day to about 1650 kcal/day, about 1300 kcal/day to about 1625 kcal/day, about 1300 kcal/day to about 1600 kcal/day, about 1300 kcal/day to about 1575 kcal/day, about 1300 kcal/day to about 1550 kcal/day, about 1300 kcal/day to about 1525 kcal/day, about 1300 kcal/day to about 1500 kcal/day, about 1300 kcal/day to about 1475 kcal/day, about 1300 kcal/day to about 1450 kcal/day, about 1300 kcal/day to about 1425 kcal/day, about 1300 kcal/day to about 1400 kcal/day, about 1325 kcal/day to about 2075 kcal/day, about 1350 kcal/day to about 2050 kcal/day, about 1375 kcal/day to about 2025 kcal/day, about 1400 kcal/day to about 2000 kcal/day, about 1425 kcal/day to about 1975 kcal/day, about 1450 kcal/day to about 1950 kcal/day, about 1475 kcal/day to about 1925 kcal/day, about 1500 kcal/day to about 1900 kcal/day, about 1525 kcal/day to about 1875 kcal/day, about 1550 kcal/day to about 1850 kcal/day, about 1575 kcal/day to about 1825 kcal/day, about 1600 kcal/day to about 1800 kcal/day, about 1625 kcal/day to about 1775 kcal/day, about 1650 kcal/day to about 1750 kcal/day, about 1675 kcal/day to about 1725 kcal/day, about 1300 kcal/day, about 1325 kcal/day, about 1350 kcal/day, about 1375 kcal/day, about 1400 kcal/day, about 1425 kcal/day, about 1450 kcal/day, about 1475 kcal/day, about 1500 kcal/day, about 1525 kcal/day, about 1550 kcal/day, about 1551 kcal/day, about 1552 kcal/day, about 1553 kcal/day, about 1554 kcal/day, about 1555 kcal/day, about 1556 kcal/day, about 1557 kcal/day, about 1558 kcal/day, about 1559 kcal/day, about 1560 kcal/day, about 1561 kcal/day, about 1562 kcal/day, about 1563 kcal/day, about 1564 kcal/day, about 1565 kcal/day, about 1566 kcal/day, about 1567 kcal/day, about 1568 kcal/day, about 1569 kcal/day, about 1570 kcal/day, about 1571 kcal/day, about 1572 kcal/day, about 1573 kcal/day, about 1574 kcal/day, about 1575 kcal/day, about 1576 kcal/day, about 1577 kcal/day, about 1578 kcal/day, about 1579 kcal/day, about 1580 kcal/day, about 1581 kcal/day, about 1582 kcal/day, about 1583 kcal/day, about 1584 kcal/day, about 1585 kcal/day, about 1586 kcal/day, about 1587 kcal/day, about 1588 kcal/day, about 1589 kcal/day, about 1590 kcal/day, about 1591 kcal/day, about 1592 kcal/day, about 1593 kcal/day, about 1594 kcal/day, about 1595 kcal/day, about 1596 kcal/day, about 1597 kcal/day, about 1598 kcal/day, about 1599 kcal/day, about 1600 kcal/day, about 1601 kcal/day, about 1602 kcal/day, about 1603 kcal/day, about 1604 kcal/day, about 1605 kcal/day, about 1606 kcal/day, about 1607 kcal/day, about 1608 kcal/day, about 1609 kcal/day, about 1610 kcal/day, about 1611 kcal/day, about 1612 kcal/day, about 1613 kcal/day, about 1614 kcal/day, about 1615 kcal/day, about 1616 kcal/day, about 1617 kcal/day, about 1618 kcal/day, about 1619 kcal/day, about 1620 kcal/day, about 1621 kcal/day, about 1622 kcal/day, about 1623 kcal/day, about 1624 kcal/day, about 1625 kcal/day, about 1626 kcal/day, about 1627 kcal/day, about 1628 kcal/day, about 1629 kcal/day, about 1630 kcal/day, about 1631 kcal/day, about 1632 kcal/day, about 1633 kcal/day, about 1634 kcal/day, about 1635 kcal/day, about 1636 kcal/day, about 1637 kcal/day, about 1638 kcal/day, about 1639 kcal/day, about 1640 kcal/day, about 1641 kcal/day, about 1642 kcal/day, about 1643 kcal/day, about 1644 kcal/day, about 1645 kcal/day, about 1646 kcal/day, about 1647 kcal/day, about 1648 kcal/day, about 1649 kcal/day, about 1650 kcal/day, about 1675 kcal/day, about 1700 kcal/day, about 1725 kcal/day, about 1750 kcal/day, about 1775 kcal/day, about 1800 kcal/day, about 1825 kcal/day, about 1850 kcal/day, about 1875 kcal/day, about 1900 kcal/day, about 1925 kcal/day, about 1950 kcal/day, about 1975 kcal/day, about 2000 kcal/day, about 2025 kcal/day, about 2050 kcal/day, about 2075 kcal/day, or about 2100 kcal/day to the subject upon ingestion of the ketogenic food composition by the subject. The ketogenic food composition may provide about 1600 kcal/day to the subject upon ingestion of the food composition by the subject. The ketogenic food composition may provide 1600 kcal/day to the subject upon ingestion of the ketogenic food composition by the subject.

The ketogenic food composition may be the sole source of calories for the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years.

In some embodiments, the ketogenic food composition may include a plurality of ketogenic food compositions. The plurality of ketogenic food compositions may be ingested daily by a subject. The plurality of ketogenic food compositions may be ingested daily by the subject such that the total ratio (i.e., representing the ratio of fat to a combination of protein and carbohydrate for an entire day) may be about 2.5:1 to about 4.5:1, about 2.6:1 to about 4.5:1, about 2.7:1 to about 4.5:1, about 2.8:1 to about 4.5:1, about 2.9:1 to about 4.5:1, about 3.0 to about 4.5:1, about 2.5:1 to about 4.4:1, about 2.5:1 to about 4.3:1, about 2.5:1 to about 4.2:1, about 2.5:1 to about 4.1:1, about 2.5:1 to about 4.0:1, about 2.6:1 to about 4.4:1, about 2.7:1 to about 4.3:1, about 2.8:1 to about 4.2:1, about 2.9:1 to about 4.1:1, or about 3.0:1 to about 4.0:1. In other embodiments, the plurality of ketogenic food compositions may be ingested daily by the subject such that the total ratio may be about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, or about 4.5:1. In other embodiments, the plurality of ketogenic food compositions may be ingested daily by the subject such that this total ratio may be about 4.0:1 or about 3.0:1. In still other embodiments, the plurality of ketogenic food compositions may be ingested daily by the subject such that this total ratio may be 4.0:1 or 3.0:1.

The plurality of ketogenic food compositions may be five ketogenic food compositions. The plurality of ketogenic food compositions may be five ketogenic food compositions ingested daily by the subject. The five ketogenic food compositions may be ingested daily by the subject such that that the total ratio (i.e., representing the ratio of fat to a combination of protein and carbohydrate for an entire day) may be about 2.5:1 to about 4.5:1, about 2.6:1 to about 4.5:1, about 2.7:1 to about 4.5:1, about 2.8:1 to about 4.5:1, about 2.9:1 to about 4.5:1, about 3.0 to about 4.5:1, about 2.5:1 to about 4.4:1, about 2.5:1 to about 4.3:1, about 2.5:1 to about 4.2:1, about 2.5:1 to about 4.1:1, about 2.5:1 to about 4.0:1, about 2.6:1 to about 4.4:1, about 2.7:1 to about 4.3:1, about 2.8:1 to about 4.2:1, about 2.9:1 to about 4.1:1, or about 3.0:1 to about 4.0:1. In other embodiments, the five ketogenic food compositions may be ingested daily by the subject such that that the total ratio may be about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, or about 4.5:1. In other embodiments, the five ketogenic food compositions may be ingested daily by the subject such that that this total ratio may be about 4.0:1 or about 3.0:1. In still other embodiments, the five ketogenic food compositions may be ingested daily by the subject such that that this total ratio may be 4.0:1 or 3.0:1.

In some embodiments, the plurality of ketogenic food compositions may be ingested by the subject in a cyclical fashion. In this cycle, X ketogenic food compositions (e.g., 1, 2, 3, 4, 5, 6, and so forth) may be ingested by the subject in Y amount of time (e.g., days, weeks, months, and years). Upon completion of one cycle, another cycle is begun by the subject. This cycle may include a daily cycle, a weekly cycle, a monthly cycle, or a yearly cycle.

The daily cycle may be at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, or 90 days. The daily cycle may be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, or 90 days. The daily cycle may be about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, or 90 days. The daily cycle may be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, or 90 days.

The weekly cycle be at least about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. The weekly cycle may be at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. The weekly cycle may be about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. The weekly cycle may be 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. The weekly cycle may be about 4 weeks or about 8 weeks. The weekly cycle may be 4 weeks or 8 weeks.

The monthly cycle may be at least about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months. The monthly cycle may be at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months. The monthly cycle may be about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months. The monthly cycle may be 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months. The monthly cycle may be about 1 month or about 2 months. The monthly cycle may be 1 month or 2 months.

The yearly cycle may be at least about 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years. The yearly cycle may be at least 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years. The yearly cycle may be about 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years. The yearly cycle may be 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years.

In some embodiments, the cycle may be a 5-week cycle such that five ketogenic food compositions are ingested by the subject per day, and thus, 175 ketogenic food compositions are ingested by the subject in the 5 weeks. Upon completion of this cycle, the cycle is begun again by the subject such that another 175 ketogenic food compositions are ingested by the subject at the rate of five ketogenic food compositions per day.

The ration for each ketogenic food composition of the plurality of ketogenic food compositions may be about may about 2.5:1 to about 11.0:1, about 2.5:1 to about 10.5:1, about 2.5:1 to about 10.0:1, about 2.5:1 to about 9.5:1, about 2.5:1 to about 9.0:1, about 2.5:1 to about 8.5:1, about 2.5:1 to about 8.0:1, about 2.5:1 to about 7.5:1, about 2.5:1 to about 7.0:1, about 2.5:1 to about 6.5:1, about 2.5:1 to about 6.0:1, about 2.5:1 to about 5.5:1, about 2.5:1 to about 5.0:1, about 2.5:1 to about 4.9:1, about 2.5:1 to about 4.8:1, about 2.5:1 to about 4.7:1, about 2.5:1 to about 4.6:1, about 2.5:1 to about 4.5:1, 2.5:1 to about 4.4:1, about 2.5:1 to about 4.3:1, about 2:5:1 to about 4.2:1, about 2.5:1 to about 4.1:1, about 2.5:1 to about 4.0:1, about 2.6:1 to about 11.0:1, about 2.7:1 to about 11.0:1, about 2.8:1 to about 11.0:1, about 2.9:1 to about 11.0:1, about 3.0:1 to about 11.0:1, about 3.1:1 to about 11.0:1, about 3.2:1 to about 11.0:1, about 3.3:1 to about 11.0:1, about 3.4:1 to about 11.0:1, about 3.5:1 to about 11.0:1, about 3.6:1 to about 11.0:1, about 3.7:1 to about 11.0:1, about 3.8:1 to about 11.0:1 about 3.9:1 to about 11.0:1, about 4.0:1 to about 11.0:1, about 4.1:1 to about 11.0:1, about 4.2:1 to about 11.0:1, about 4.3:1 to about 11.0:1, about 4.4:1 to about 11.0:1, about 4.5:1 to about 11.0:1, about 4.6:1 to about 11.0:1, about 4.7:1 to about 11.0:1, about 4.8:1 to about 11.0:1, about 4.9:1 to about 11.0:1, about 5.0:1 to about 11.0:1, about 5.5:1 to about 11.0:1, about 6.0:1 to about 11.0:1, about 6.5:1 to about 11.0:1, about 7.0:1 to about 11.0:1, about 7.5:1 to about 11.0:1, about 8.0:1 to about 11.0:1, about 8.5:1 to about 11.0:1, about 9.0:1 to about 11.0:1, about 9.5:1 to about 11.0:1, about 10.0:1 to about 11.0:1. In some embodiments, the ratio for each ketogenic food composition of the plurality of ketogenic food compositions may be about 2.5:1 to about 4.5:1, about 2.6:1 to about 4.5:1, about 2.7:1 to about 4.5:1, about 2.8:1 to about 4.5:1, about 2.9:1 to about 4.5:1, about 3.0 to about 4.5:1, about 2.5:1 to about 4.4:1, about 2.5:1 to about 4.3:1, about 2.5:1 to about 4.2:1, about 2.5:1 to about 4.1:1, about 2.5:1 to about 4.0:1, about 2.6:1 to about 4.4:1, about 2.7:1 to about 4.3:1, about 2.8:1 to about 4.2:1, about 2.9:1 to about 4.1:1, or about 3.0:1 to about 4.0:1. In other embodiments, the ratio for each ketogenic food composition of the plurality of ketogenic food compositions may be about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, or about 4.5:1. In still other embodiments, the ratio for each ketogenic food composition of the plurality of ketogenic food compositions may be 4.0:1 or 3.0:1.

The ketogenic food composition may be frozen, hyalinized, vacuum-packed, or any combination thereof prior to ingestion of the ketogenic food composition by the subject. The ketogenic food composition may be frozen prior to ingestion of the food composition by the subject.

a. Ketogenesis

The ketogenic food composition promotes ketogenesis in the subject in need thereof. The ketogenic food composition increases ketogenesis in the subject relative to ketogenesis in the subject prior to ingestion of the ketogenic food composition.

Ketogenesis is a process by which ketone bodies are produced as a result of fatty acid breakdown. The ketone bodies include acetoacetate, acetone, and β-hydroxybutyrate. Ketone bodies may be an alternative energy source for cells when glucose is unavailable or present at low levels. Specifically, ketone bodies are produced in the mitochondria of liver cells in response to low glucose levels in the blood or the exhaustion of cellular carbohydrate stores (e.g., glycogen).

Cancer cells, unlike non-cancerous cells, are largely unable to utilize ketone bodies as an energy source, and thus, are starved in the absence of or in the presence of low levels of glucose. About 95% of cancers rely on the metabolism of glucose as an energy source and thus, cannot utilize ketone bodies as an energy source. In particular, glioblastoma multiforme relies on glucose metabolism as an energy source and cannot utilize ketone bodies as an energy source. Additionally, ketone bodies may sensitize cancer cells to radiation.

Accordingly, the ketogenic food composition, by promoting ketogenesis in the subject, shifts the balance of energy sources in the subject such that ketone bodies are the predominant energy source for cells. This, in turn, removes the energy source for cancer cells (e.g., glioblastoma multiforme cells), thereby preventing or slowing growth of the cancer.

Furthermore, the ketogenic food composition, by promoting ketogenesis in the subject, lowers or reduces the availability of glucose in the blood, which is advantageous in diseases in which blood glucose levels are abnormally high, for example, type 2 diabetes. In turn, this lowering of glucose levels in the blood reduces insulin production, which is stimulated by high or excess blood glucose levels.

b. Fat

The ketogenic food composition includes fat. The fat may include saturated fat. The fat may be derived from dairy products, oils, and oil-based products, for example, but not limited to, heavy cream, mascarpone, sour cream, crème freche, butter, mayonnaise, high fat content fish and meats, low carbohydrate vegetables and fruits, high-fat containing nuts, and any combination thereof.

High fat content fish and meat may include, but are not limited to, crab meat, prosciutto, chicken thighs, salmon, catfish, mackerel, bacon, ham, pork shoulder, pork chops, ground pork, pork frankfurters, and any combination thereof. High-fat containing nuts may include, but are not limited to, Brazil nuts, Pecan nuts, walnuts, almonds, hazelnuts, and any combination thereof.

The ketogenic food composition may contain about 10 grams (g) of fat to about 100 g of fat, about 10 g of fat to about 95 g of fat, about 10 g of fat to about 90 g of fat, about 10 g of fat to about 85 g of fat, about 10 g of fat to about 80 g of fat, about 10 g of fat to about 75 g of fat, about 10 g of fat to about 70 g of fat, about 10 g of fat to about 65 g of fat, about 10 g of fat to about 60 g of fat, about 15 g of fat to about 100 g of fat, about 20 g of fat to about 100 g of fat, about 25 g of fat to about 100 g of fat, about 15 g of fat to about 80 g of fat, or about 20 g of fat to about 60 g of fat.

c. Protein

The ketogenic food composition includes protein. The ketogenic food composition may contain about 1 g of protein to about 20 g of protein, about 1 g of protein to about 18 g of protein, about 1 g of protein to about 16 g of protein, about 1 g of protein to about 14 g of protein, about 1 g of protein to about 12 g of protein, about 1 g of protein to about 10 g of protein, about 1 g of protein to about 9 g of protein, about 1 g of protein to about 8 g of protein, about 1 g of protein to about 7 g of protein, about 1 g of protein to about 6 g of protein, about 1 g of protein to about 5 g of protein, about 1 g of protein to about 4 g of protein, about 2 g of protein to about 20 g of protein, about 3 g of protein to about 20 g of protein, about 2 g of protein to about 18 g of protein, or about 3 g of protein to about 16 g of protein.

Like the fat sources above, particular protein sources are used in the ketogenic composition. Protein sources may include, but are not limited to, fish, meat, and nuts. Fish and meat may include, but are not limited, crab meat, prosciutto, chicken thighs, salmon, catfish, mackerel, bacon, ham, pork shoulder, pork chops, ground pork, pork frankfurters, and any combination thereof. Nuts may include, but are not limited to, Brazil nuts, Pecan nuts, walnuts, almonds, hazelnuts, and any combination thereof.

d. Carbohydrate

The ketogenic food composition includes carbohydrate. The ketogenic food composition may contain about 0.5 g of carbohydrate to about 8 g of carbohydrate, about 0.8 g of carbohydrate to about 8 g of carbohydrate, about 1 g of carbohydrate to about 8 g of carbohydrate, about 1.2 g of carbohydrate to about 8 g of carbohydrate, about 1.5 g of carbohydrate to about 8 g of carbohydrate, about 0.5 g of carbohydrate to about 7 g of carbohydrate, about 0.5 g of carbohydrate to about 6 g of carbohydrate, about 0.5 g of carbohydrate to about 5.5 g of carbohydrate, about 0.8 g of carbohydrate to about 7 g of carbohydrate, about 1 g of carbohydrate to about 6 g of carbohydrate, or about 1.2 g of carbohydrate to about 5.5 g of carbohydrate.

The ketogenic food composition may contain less than or equal to about 25 g carbohydrate per day, 24 g carbohydrate per day, 23 g carbohydrate per day, 22 g carbohydrate per day, 21 g carbohydrate per day, 20 g carbohydrate per day, 19 g carbohydrate per day, 18 g carbohydrate per day, 17 g carbohydrate per day, 16 g carbohydrate per day, 15 g carbohydrate per day, 14 g carbohydrate per day, 13 g carbohydrate per day, 12 g carbohydrate per day, 11 g carbohydrate per day, 10 g carbohydrate per day, 9 g carbohydrate per day, 8 g carbohydrate per day, 7 g carbohydrate per day, 6 g carbohydrate per day, or 5 g carbohydrate per day. The ketogenic food composition may contain less than or equal to about 20 g carbohydrate per day. The ketogenic food composition may contain less than or equal to about 10 g carbohydrate per day.

In some embodiments, the ketogenic food composition may contain less than or equal to about 25 g carbohydrate per day, 24 g carbohydrate per day, 23 g carbohydrate per day, 22 g carbohydrate per day, 21 g carbohydrate per day, 20 g carbohydrate per day, 19 g carbohydrate per day, 18 g carbohydrate per day, 17 g carbohydrate per day, 16 g carbohydrate per day, 15 g carbohydrate per day, 14 g carbohydrate per day, 13 g carbohydrate per day, 12 g carbohydrate per day, 11 g carbohydrate per day, 10 g carbohydrate per day, 9 g carbohydrate per day, 8 g carbohydrate per day, 7 g carbohydrate per day, 6 g carbohydrate per day, or 5 g carbohydrate per day when the subject is suffering from the obesity-related disorder. The ketogenic food composition may contain less than or equal to about 20 g carbohydrate per day when the subject is suffering from the obesity-related disorder, which is described below in more detail.

In other embodiments, the ketogenic food composition may contain less than or equal to about 25 g carbohydrate per day, 24 g carbohydrate per day, 23 g carbohydrate per day, 22 g carbohydrate per day, 21 g carbohydrate per day, 20 g carbohydrate per day, 19 g carbohydrate per day, 18 g carbohydrate per day, 17 g carbohydrate per day, 16 g carbohydrate per day, 15 g carbohydrate per day, 14 g carbohydrate per day, 13 g carbohydrate per day, 12 g carbohydrate per day, 11 g carbohydrate per day, 10 g carbohydrate per day, 9 g carbohydrate per day, 8 g carbohydrate per day, 7 g carbohydrate per day, 6 g carbohydrate per day, or 5 g carbohydrate per day when the subject is suffering from the cancer. The ketogenic food composition may contain less than or equal to about 10 g carbohydrate per day when the subject is suffering from the cancer (e.g., glioblastoma multiforme, which is described in more detail below).

Typical sources of carbohydrates for the ketogenic food composition may include low level carbohydrate sources. Low level carbohydrate sources include, but are not limited to, vegetables and fruits. Vegetables and fruits providing a low level of carbohydrates may include, but are not limited to, raspberries, strawberries, blueberries, apricots, leeks, cucumbers, lettuce (e.g., Iceberg lettuce), spinach, cauliflower, celery, Chinese cabbage, mushrooms, avocado, olives, cucumber, broccoli, green pepper, red pepper, asparagus, tomato, onion, eggplant, zucchini, spring onions, carrots, watercress, and any combination thereof.

### 2. METHOD OF TREATMENT

Provided herein is a method of treating a disease in a subject in need thereof. The disease may be the obesity-related disorder (e.g., type 2 diabetes, obstructive sleep apnea, polycystic ovarian syndrome, and metabolic syndrome) or the cancer (e.g., glioblastoma multiforme) as described below in more detail. The method may include administering the ketogenic food composition to the subject and ingestion of the ketogenic food composition by the subject. The ketogenic food composition may be ingested by the subject at a particular schedule over a particular period of time as discussed above.

Over time and participation of the subject in the method of treatment, ingestion (leading to digestion) of the ketogenic food composition may reduce one or more symptoms of an obesity-related disorder in the subject. Ingestion of the ketogenic food composition may reverse progression of the obesity-related disorder in the subject. Ingestion of the ketogenic food composition may slow progression of the obesity-related disorder in the subject. Ingestion of the ketogenic food composition may prevent onset of the obesity-related disorder in the subject. Ingestion of the ketogenic food composition may prevent progression of the obesity-related disorder in the subject. The obesity-related disorder may be, but is not limited to, type 2 diabetes, obstructive sleep apnea, polycystic ovarian syndrome, metabolic syndrome, and any combination thereof.

Ingestion of the ketogenic food composition may reverse progression of a cancer in the subject. Ingestion of the ketogenic food composition may slow progression of the cancer in the subject. Ingestion of the ketogenic food composition may prevent the cancer in the subject. Ingestion of the ketogenic food composition may prevent progression of the cancer in the subject. Ingestion of the ketogenic food composition may prevent metastasis of the cancer in the subject. Ingestion of the ketogenic food composition may slow metastasis of the cancer in the subject. The cancer may be, but is not limited to, glioblastoma multiforme.

The method of treatment may reduce one or more symptoms of the disease in the subject relative to one or more symptoms of the disease in the subject prior to initiation of the method of treatment. The method of treatment may slow the progression of the disease in the subject relative to a progression of the disease in the subject prior to initiation of the method of treatment. The method of treatment may reverse progression of the disease in the subject. The method of treatment may cause remittance of the disease in the subject. The method of treatment may prevent progression of the disease in the subject. The method of treatment may prevent the onset of the disease in the subject. The disease may be the obesity-related disorder (e.g., type 2 diabetes, obstructive sleep apnea, polycystic ovarian syndrome, and metabolic syndrome) or the cancer (e.g., glioblastoma multiforme). One or more of these foregoing improvements provided by the method of treatment may continue in the subject after termination of the method of treatment.

Accordingly, the benefits provided to the subject by the method of treatment outlast (i.e., extend beyond) the duration of the method of treatment. The benefits provided to the subject by the method of treatment may extend for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years beyond the termination of the method of treatment.

a. Obesity-Related Disorder

The method of treatment may include treating the obesity-related disorder in the subject. This method may include administering the ketogenic food composition to the subject. The ketogenic food composition may have the ratio of 3:1 or 4:1. The obesity-related disorder may be type 2 diabetes, obstructive sleep apnea, polycystic ovarian syndrome, metabolic syndrome, or any combination thereof.

The method of treatment may reduce a level of obesity in the subject relative to a level of obesity in the subject prior to initiation of the method of treatment. The level of obesity may be a body mass index (BMI) of the subject. The method of treatment may reduce the body mass index of the subject relative to a body mass index of the subject prior to initiation of the method of treatment. The method of treatment may reduce the body mass index of the subject by at least about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% relative to the body mass index of the subject prior to initiation of the method of treatment. The foregoing reduction in the body mass index of the subject afforded by the method of treatment may remain reduced in subject after termination of the method of treatment.

Accordingly, the subject after ending the method of treatment may continue to have a lower BMI relative to the BMI of the subject prior to initiation of the method of treatment for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years after termination of the method of treatment.

(1) Type 2 Diabetes

The method of treatment may include treating type 2 diabetes in the subject. The method may include administering the ketogenic food composition to the subject. The ketogenic food composition may have the ratio of 3:1 or 4:1. The method of treatment may reduce one or more symptoms of type 2 diabetes in the subject relative to one or more symptoms of type 2 diabetes in the subject prior to initiation of the method of treatment. The method of treatment may slow the progression of type 2 diabetes in the subject relative to a progression of type 2 diabetes in the subject prior to initiation of the method of treatment. The method of treatment may reverse the progression of type 2 diabetes in the subject. The method of treatment may cause remittance of type 2 diabetes in the subject. The method of treatment may prevent the progression of type 2 diabetes in the subject. The method of treatment may prevent the onset of type 2 diabetes in the subject. The method of treatment may reverse insulin resistance in the subject. The method of treatment may reverse hyperinsulinema in the subject.

The method of treatment may improve a blood glucose level, an insulin level, a glycosylated hemoglobin (HbA1C) level, or any combination thereof in the subject relative to a blood glucose level, an insulin level, and/or a glycosylated hemoglobin (HbA1C) level in the subject prior to initiation of the method of treatment.

The method of treatment may improve the blood glucose level of the subject relative to the blood glucose level of the subject prior to initiation of the method of treatment. This improvement in the blood glucose level of the subject may include the subject having a fasting blood glucose level of less than about 100 mg/dL. This improvement in the blood glucose level of the subject may include the subject maintaining a fasting blood glucose level of less than about 100 mg/dL in the absence of one or more diabetic medications.

In other embodiments, this improvement in the blood glucose level of the subject may extend beyond the termination of the method of treatment. This improvement in the blood glucose level of the subject may extend beyond the termination of the method of treatment such that the subject maintains a fasting blood glucose level of less than about 100 mg/dL after termination of the method of treatment. This improvement in the blood glucose level of the subject may extend beyond the termination of the method of treatment such that the subject maintains a fasting blood glucose level of less than about 100 mg/dL for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years after termination of the method of treatment.

The method of treatment may improve the insulin level of the subject relative to the insulin level of the subject prior to initiation of the method of treatment. This improvement may include reducing the insulin level of the subject prior to initiation of the method of treatment by about 6.0-fold, 5.5-fold, 5.0-fold, 4.5-fold, 4.0-fold, 3.5-fold, 3.0-fold, 2.5-fold, 2.0-fold, or 1.5-fold. This improvement in the insulin level of the subject may extend beyond the termination of the method of treatment.

The method of treatment may improve the HbA1C level of the subject relative to the HbA1C level of the subject prior to initiation of the method of treatment. The HbA1C level reflects average blood glucose levels over the past three months, and thus, serves as a marker of blood glucose control over a longer period of time. This improvement in the HbA1C level of the subject may include the subject having a fasting HbA1C level of less than about 5.7%. A fasting HbA1C level of less than about 5.7% is normal (i.e., non-diabetic or non-pre-diabetic). This improvement in the HbA1C level of the subject may include the subject having a fasting HbA1C level of less than about 5.7% in the absence of one or more diabetic medications.

In other embodiments, this improvement in the HbA1C level of the subject may extend beyond the termination of the method of treatment. This improvement in the HbA1C level of the subject may extend beyond the termination of the method of treatment such that the subject has a fasting HbA1C level of less than about 5.7% after termination of the method of treatment. This improvement in the HbA1C level of the subject may extend beyond the termination of the method of treatment such that the subject has a fasting HbA1C level of less than about 5.7% for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years after termination of the method of treatment.

(2) Obstructive Sleep Apnea

The method of treatment may include treating obstructive sleep apnea in the subject. The method may include administering the ketogenic food composition to the subject. The ketogenic food composition may have the ratio of 3:1 or 4:1. The method of treatment may reduce one or more symptoms of obstructive sleep apnea in the subject relative to one or more symptoms of obstructive sleep apnea in the subject prior to initiation of the method of treatment. The method of treatment may slow the progression of obstructive sleep apnea in the subject relative to a progression of obstructive sleep apnea in the subject prior to initiation of the method of treatment. The method of treatment may reverse the progression of obstructive sleep apnea in the subject. The method of treatment may cause remittance obstructive sleep apnea in the subject. The method of treatment may prevent progression of obstructive sleep apnea in the subject. The method of treatment may prevent onset of obstructive sleep apnea in the subject.

The method of treatment may improve an apnea/hyponea index (AHI) of the subject relative to an AHI of the subject prior to initiation of the method of treatment. The method may improve an AHI of the subject by at least about 5%, about 10%, about 15%, about 20%, about 25%, about 30%, about 35%, about 40%, about 45%, about 50%, about 55%, about 60%, about 65%, about 70%, or about 75% relative to the AHI of the subject prior to initiation of the method of treatment. This improvement in the AHI of the subject may extend beyond the termination of the method of treatment.

The method of treatment may reduce the AHI of the subject to less than or equal to 5 (i.e., to normal). The AHI of the subject may remain less than or equal to 5 (i.e., normal) for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, or 25 years after termination of the method of treatment.

In some embodiments, if the subject is undergoing continuous positive air pressure (CPAP) treatment prior to initiation of the method of treatment described herein, the method may improve the AHI of the subject such that obstructive sleep apnea is remitted and the CPAP treatment may be discontinued.

(3) Polycystic Ovarian Syndrome

The method of treatment may include treating polycystic ovarian syndrome in the subject. The method may include administering the ketogenic food composition to the subject. The ketogenic food composition may have the ratio of 3:1 or 4:1. The method of treatment may reduce one or more symptoms of polycystic ovarian syndrome in the subject relative to one or more symptoms of polycystic ovarian syndrome in the subject prior to initiation of the method of treatment. The method of treatment may slow the progression of polycystic ovarian syndrome in the subject relative to a progression of polycystic ovarian syndrome in the subject prior to initiation of the method of treatment. The method of treatment may reverse the progression of polycystic ovarian syndrome in the subject. The method of treatment may cause remittance of polycystic ovarian syndrome in the subject. The method of treatment may prevent progression of polycystic ovarian syndrome in the subject. The method of treatment may prevent onset of polycystic ovarian syndrome in the subject.

(4) Metabolic Syndrome

The method of treatment may include treating metabolic syndrome in the subject. The method may include administering the ketogenic food composition to the subject. The ketogenic food composition may have the ratio of 3:1 or 4:1. The method of treatment may reduce one or more symptoms of metabolic syndrome in the subject relative to one or more symptoms of metabolic syndrome in the subject prior to initiation of the method of treatment. The method of treatment may slow the progression of metabolic syndrome in the subject relative to a progression of metabolic syndrome in the subject prior to initiation of the method of treatment. The method of treatment may reverse the progression of metabolic syndrome in the subject. The method of treatment may cause remittance of metabolic syndrome in the subject. The method of treatment may prevent progression of metabolic syndrome in the subject. The method of treatment may prevent onset of metabolic syndrome in the subject.

b. Glioblastoma Multiforme

The method of treatment may include treating cancer in the subject. The method may include administering the ketogenic food composition to the subject for treating cancer. The cancer may be glioblastoma multiforme (GBM). The ketogenic food composition may have the ratio of 4:1. Ingestion of the ketogenic food composition may reduce a size of a tumor in the subject relative to a size of the tumor prior to ingestion of the ketogenic food composition by the subject. Ingestion of the ketogenic food composition may slow growth of the tumor in the subject. Ingestion of the ketogenic food composition may prevent growth of the tumor in the subject. The tumor may be from a glioblastoma multiforme cancer.

The method of treating GBM may be an adjuvant therapy. The method of treating GBM may be an adjuvant therapy to a radiation therapy, a chemotherapy, or a combination thereof.

The method of treatment may reduce one or more symptoms of GBM in the subject relative to one or more symptoms of GBM in the subject prior to initiation of the method of treatment. The method of treatment may slow the progression of GBM in the subject relative to a progression of GBM in the subject prior to initiation of the method of treatment. The method of treatment may reverse progression of GBM in the subject. The method of treatment may cause remittance of GBM in the subject. The method of treatment may prevent progression of GBM in the subject. The method of treatment may prevent onset of GBM in the subject.

The method of treatment may reduce a size of a GBM tumor in the subject relative to a size of the GBM tumor in the subject prior to initiation of the method of treatment. The method of treatment may slow the growth of the GBM tumor in the subject. The method of treatment may prevent the growth of the GBM tumor in the subject.

The method of treatment may prevent metastasis from the GBM in the subject. The method of treatment may slow metastasis from the GBM in the subject.

The method of treatment may increase the survival of GBM in the subject by at least 1.5-fold, about 2.0-fold, about 2.5-fold, about 3.0-fold, about 3.5-fold, about 4.0-fold, about 4.5-fold, about 5.0-fold, about 5.5-fold, about 6.0-fold, about 6.5-fold, about 7.0-fold, about 7.5-fold, or about 8.0-fold relative to a subject having GBM that did not receive the method of treatment.

### 3. METHOD OF MANUFACTURE

Also provided herein is a method of manufacturing the ketogenic food composition. The method of manufacturing may include preparing the ketogenic food composition. Preparing the ketogenic food composition may include freezing the ketogenic food composition, hyalinizing the ketogenic food composition, vacuum-packing the ketogenic food composition, or any combination thereof. Preparing may also include placing the ketogenic food composition in one or more containers in which the ketogenic food composition is stored until consumption of the ketogenic food composition by the subject. The one or more containers may be included in a kit as described in more detail below.

### 4. KIT

Also provided herein is a kit including the ketogenic food composition. The kit may be used in the method of the treatment described above. The kit may include the one or more containers in which the ketogenic food composition may be stored until consumption of the ketogenic food composition by the subject.

The kit according to the present disclosure may also include instructions for carrying out the method of treatment. Instructions included in the kit of the present disclosure may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

In another aspect, the disclosure provides for a kit comprising, consisting essentially of, or consisting of any of the meals or dietary packages disclosed herein. In an aspect, the kit includes any of the meals or dietary packages described in Examples 1-11. In another aspect, the kit provides for the meals or dietary packages described in Examples 1-11, applied in a manner that is consistent with the methodology of these examples and figures. In another aspect, the kit provides instructions or guidance regarding the use of the meals, dietary packages, or methods described herein.

In an aspect, the kit includes instructions describing the methodology described herein. In another aspect, the kit includes instructions describing the methodology set forth in any of Examples 1-11. In an aspect, the instructions are included with the kit, separate from the kit, in the kit, or are included on the kit packaging.

The present invention has multiple aspects, illustrated by the following non-limiting examples.

### 5. EXAMPLES

### Example 1

### Ketogenic Food Compositions for One Day

As described above, the ketogenic food composition may be the plurality of ketogenic food compositions, and more specifically, five ketogenic food compositions that are ingested by the subject in a single day. Below are five ketogenic food compositions that were ingested by subjects in the single day and the sole source of calories to the subject in this single day.

Breakfast.

The ingredients were 70 g raspberries, 3 tablespoons (tbsp.) whipping cream, 10 g Brazil nuts, and 1 sachet of the sweetener sold under the name SPLENDA. This breakfast was prepared by whipping the cream and cutting or grating the Brazil nuts into small pieces. The whipped cream and small pieces of Brazil nuts were mixed with the raspberries and the sweetener. This breakfast provided 256 calories and contained 5.3 g of carbohydrates, 25.1 g fat, and 3.5 g protein. The ratio was 2.9:1.

Am Snack.

The am snack was leek soup. The ingredients were 40 g leeks, 25 g butter, and 0.25 of a chicken bouillon cube. This am snack was prepared by cutting the leeks into small strips. The butter was melted and the strips were simmered in the melted butter for about 2 minutes. 100 milliliters (mL) of water and the chicken bouillon cube were added to the strips and butter. This resulting mixture was brought to a boil and boiled for about 10 minutes. The volume was then brought up to 150 mL and salt was added to taste. This am snack provided 173 calories and contained 5.5 g carbohydrates, 20.5 g fat, and 1 g protein. The ratio was 3.15:1.

Lunch.

Lunch was a crab salad. The ingredients were 50 g crab meat, 50 g pickled cucumber, 100 g iceberg lettuce, 10 g fresh dill, 0.5 teaspoon (tsp) mustard, 60 g mayonnaise, and 0.5 tsp lemon juice. This lunch was prepared by cutting the crab meat and iceberg lettuce into small strips. The cucumber was coarsely grated. The dill was finely chopped. The crab meat was then sprinkled with the lemon juice. The crab meat, pickled cucumber, iceberg lettuce, dill, mustard powder, and lemon juice were then mixed together. To the resulting mixture, mayonnaise was added and mixed in. The lunch was frozen and then defrosted and salted to taste prior to consumption. This lunch provided 483 calories and contained 4.6 g carbohydrates, 47.9 g fat, and 10.6 g protein. The ratio was 3.2:1

Pm Snack.

The pm snack was a prosciuotto shake. The ingredients were 0.5 ounces (oz.) prosciutto, 40 g crème freche, 2 tbsp whipping cream, and a dash of Old boys spice. This pm snake was prepared by cutting the prosciutto into small pieces and mixing these small pieces with the crème freche, spice, and whipping cream. The volume of the resulting mixture was brought up to 150 mL with warm water and then mixed again. This pm snack provided 211 calories and contained 1.4 g carbohydrates, 19.3 g fat, and 4.7 g protein. The ratio was 3.12:1.

Dinner.

Dinner was chicken thighs in spinach sauce. The ingredients were 60 g chicken thigh, 2 tbsp oil, 100 g spinach, and 50 g crème freche. This dinner was prepared by cutting the chicken thigh into slices. The slices were sprinkled with salt and fried in the oil on high heat for about 2 minutes on each side of the slices. The slices were then put aside and kept warm. The heat was lowered and the spinach was sautéed until it wilted. The spinach was removed from the heat and mixed with the slices and crème freche. This dinner provided 494 calories and contained 2.2 g carbohydrate, 56.3 g fat, and 14.9 g protein. The ratio was 3.3:1.

Summary.

The five ketogenic food compositions (i.e., meals) above provided 1618 total calories for the day and together contained 20 g carbohydrates, 169.1 g fat, and 34.7 g protein.

### Example 2

### Ketogenic Food Compositions for Eight Days

As described above, the ketogenic food composition may be the plurality of ketogenic food compositions, and more specifically, five ketogenic food compositions per day that were ingested by subjects for eight days. Below are ketogenic food compositions that were ingested by subjects for 8 days (i.e., 5 ketogenic compositions per day) and the sole source of calories to the subject in each day.

Day 1.

The total calories for day 1 were 1572 calories. The total carbohydrates for day 1 were 19.6 g carbohydrates. The total fat for day 1 were 160.5 g fat. The total protein for day 1 was 26.6 g protein.

Breakfast—

Mascarpone with strawberries

Ingredients:

Strawberries 50 g

Mascarpone 50 g

Whipping cream 1 tbsp

Splenda 1 sachet

Method:

Mixed pieces of strawberries with Splenda and mascarpone and added whipped cream.

The meal contained 283 calories, 4.6 g carbohydrates, 18.7 g fat, and 0.8 g protein. The ratio was 3.8:1.

Am Snack—

Sour cream and fresh dill

Ingredients:

Sour cream 50 g

Dill fresh 10 g

Whipping cream 1 tbsp

Method:

Mixed the creams with finely chopped dill and added salt to taste. Made up to 150 ml with water.

The meal contained 153 calories, 2.1 g carbohydrates, 14.4 g fat, and 1.9 g protein. The ratio was 3.6:1.

Lunch—

Egg, tomato and zucchini

Ingredients:

One Large egg

Onion 15 g

Tomato raw 50 g

Zucchini 50 g

Oil 2 tbsp

Butter 20 g

Method:

Peel the tomato. Sauté the onion in oil on low heat for 2-3 minutes, add thin slices of zucchini and pieces of tomato, add salt to taste, cover and cook until the vegetables are soft. Fry the egg in melted butter and serve with the vegetables.

The meal contained 477 calories, 4.6 g carbohydrates, 48.7 g fat, and 7.5 g protein. The ratio was 4.0:1.

Pm Snack—

Cauliflower soup

Ingredients:

Cauliflower 50 g

Butter 20 g

Mace ⅛ tsp

Chicken cube ¼ cube

Method:

Cauliflower was broken into small florets and boiled in salted water with mace until soft (about 10 min). The liquid was drained, but kept. The cauliflower was mashed with a fork. The butter was melted in a saucepan and the cauliflower was fried for about 1 minute. The reserved liquid was added and salt was added if needed. The resulting mixture was brought to a boil and then removed from the heat and made up to 150 ml with warm water.

This meal contained 133 calories, 2.1 g carbohydrates, 16.6 g fat, and 1.3 g protein. The ratio was 3.0:1.

Additionally, for this meal, fruit tea and 1 sachet splenda provided 3 calories and 1 g carbohydrate.

Dinner—

Chicken thigh with mushroom sauce

Ingredients:

Chicken thighs 60 g

Onion 15 g

Portabella mushrooms 50 g

Oil 2 tbsp

Crème freche 60 g

Chinese cabbage 100 g

Method:

The thighs were cut into slices. ¼ tsp salt was rubbed into the thighs and the thighs were fried on medium-high to high heat in oil for 2-3 minutes on each side. The thighs were put aside and kept warm. The onion was sautéed in the oil on low heat until translucent. Thinly sliced mushrooms were added and the resulting mixture was covered and boiled until the mushrooms were soft. The heat was switched off and the crème freche was added to the juices. The resulting mixture was mixed and the meat was then added. Pieces of the Chinese cabbage were boiled briefly (about 1 minute) in salted water and served with the meat and sauce.

This meal contained 523 calories, 5.2 g carbohydrates, 62.1 g fat, and 15.1 g protein. The ratio was 3.1:1.

Day 2.

The total calories for day 2 were 1577 calories. The total carbohydrates for day 2 were 19.2 g carbohydrates. The total fat for day 2 was 170.4 g fat. The total protein for day 2 was 34.6 g protein.

Breakfast—

Omelette with asparagus

Ingredients:

One Egg

Butter 20 g

Asparagus 50 g

Whipping cream 2 tbsp

Pepper ⅛ tsp

Method:

The asparagus was broken into small pieces and boiled in salted water until soft using just enough water to cover the vegetables. The egg, cream, pepper and salt were mixed in a bowl and the asparagus was added. This mixture was fried in the melted butter.

The meal contained 299 calories, 1.2 g carbohydrates, 34.7 g fat, and 7.9 g protein. The ratio was 3.8:1.

Additionally, breakfast included coffee as described below.

Coffee

Ingredients:

Coffee 1 tsp

Splenda 1 sachet

Whipping cream 1 tbsp

Method:

The coffee and splenda were dissolved in 150 ml boiling water and the cream was added. The cream may be whipped. This provided 97 calories, 1.1 g carbohydrates, 6 g fat, and 0.2 g protein. The ratio was 4.4:1.

Am Snack—

Dill shake II

Ingredients:

Dill (fresh) 10 g

Sour cream 6 g

Crème freche 30 g

Whipping cream 1 tbsp

Method:

Finely chopped dill was mixed with 50 ml of hot water and all the other ingredients were added. The resulting mixture was made up to 150 ml with cold water. Salt was added to taste.

This meal contained 130.4 calories, 1.1 g carbohydrates, 20.1 g fat, and 0.83 g protein. The ratio was 10.0:1.

Lunch—

Broccoli fried

Ingredients:

Broccoli 100 g

One Egg

Whipping cream 1 tbsp

Oil 2 tbsp

Method:

The florets of broccoli were covered with water and a pinch of salt was added. This was brought to a boil, boiled for 10 minutes, and drained. The egg, pinch of salt and the cream were mixed in a small bowl. Each floret was dipped in this mixture, making sure the floret was covered. The florets were fried in hot oil for about 1 minute. The remaining mixture was poured over the broccoli in the frying pan, and fried and served with the fried florets.

The meal contained 394 calories, 4.4 g carbohydrates, 39.7 g fat, and 9.1 g protein. The ratio was 2.9:1.

Additionally, for this meal, fruit tea and 1 sachet splenda provided 3 calories and 1 g carbohydrates.

Pm Snack—

Tomato salad

Ingredients:

Tomato 100 g

Spring onion 50 g

Oil (Olive) 1½ tbsp

Method:

The tomato was sliced thinly and mixed with small pieces of onions. This mixture was sprinkled with olive oil and salt was added to taste. The mixture was mixed again and stood for about 10 minutes before serving.

This meal contained 215 calories, 3.8 g carbohydrates, 20.7 g fat, and 1.7 g protein. The ratio was 3.8:1.

Dinner—

Pork with spinach sauce

Ingredients:

Pork shoulder 50 g

Spinach 100 g

Onion 30 g

Crème freche 60 g

Whipping cream 3½ tbsp

Method:

The meat was sprinkled with salt and slices of onions were placed on top of the meat. This was then wrapped tightly in aluminum foil and baked at 400° F. for about an hour. It was better to prepare meat for several portions in this way, say bake 240 g, otherwise the meat was too dry. The baked meat was divided into portions and stored in the freezer for further use.

The fresh spinach was steamed for 2-3 min until it wilted. The spinach was then mixed with crème freche and whipping cream and kept warm. The meat was taken out of the oven and the foil was carefully unwrapped. The meat and juices were added to the spinach.

This meal contained 431 calories, 3.9 g carbohydrates, 51.0 g fat, and 14.0 g protein. The ratio was 2.9:1.

Day 3.

The total calories for day 3 were 1592 calories. The total carbohydrates for day 3 were 21 g carbohydrates. The total fat for day 3 was 170.4 g fat. The total protein for day 3 was 34.6 g protein. The total ratio for day 3 was 3.06:1.

Breakfast—

Mascarpone with carrots

Ingredients:

Mascarpone 50 g

Baby carrots 30 g

Brazil Nuts 15 g

Method:

The mascarpone was mixed with finely grated carrots and chopped nuts.

The meal contained 323 calories, 3.6 g carbohydrates, 22.5 g fat, and 3.8 g protein. The ratio was 3.5:1.

Am Snack—

Vanilla shake

Ingredients:

Vanilla extract 1 tsp

Whipping cream 3 tbsp

Half and half 3 tbsp

Splenda 1 sachet

Method:

The splenda was dissolved in 100 ml boiling water. The creams and vanilla extract were then added. The mixture was mixed and made up to 150 ml with water.

This meal contained 218 calories, 1.5 g carbohydrates, 24 g fat, and 1.5 g protein. The ratio was 6.0:1.

Lunch—

Avocado with crème fresh and garlic

Ingredients:

Avocado 60 g

Lemon juice ½ tsp

Garlic powder ¼ tsp

Pepper ⅛ tsp

Olive oil 1½ tbsp

Crème freche 30 g

Method:

Avocado was scooped out the shell and cut into small pieces. Lemon juice, garlic powder, pepper, salt and oil were added and this was made into a paste (with some pieces of avocado preserved). Crème freche was added and the mixture was mixed thoroughly.

This meal contained 343 calories, 6.9 g carbohydrates, 37.9 g fat, and 2.5 g protein. The ratio was 4.0:1.

Pm Snack—

Asparagus soup

Ingredients:

Asparagus 100 g

One egg yolk

Butter 15 g

Whipping cream 1 tbsp

Method:

About ½ inch long pieces of asparagus were boiled in 100 ml of salted water until soft (about 10 min). This was liquidized. The egg yolk was whisked in and the mixture was boiled for about 30 sec. The cream was added and the mixture was made up to 150 ml with water. Salt was added to taste if necessary.

The meal contained 221 calories, 2.2 g carbohydrates, 22.8 g fat, and 5 g protein. The ratio was 3.2:1.

Additionally, with the meal, fruit tea and 1 sachet splenda provided 3 calories and 1 g carbohydrates.

Dinner—

Catfish I

Ingredients:

Catfish 100 g

Butter 30 g

Lemon juice ½ tsp

Lemon pepper ¼ tsp

Celery 50 g

Crème freche 30 g

Whipping cream 2 tbsp

Method:

The fish was sprinkled with lemon juice, lemon pepper and salt on both sides. The fish was then left for 5-10 min. The fish was fried in melted butter for 3-4 minutes on each side. The fish was put aside and kept warm. Small pieces of celery were sautéed until soft. The celery was removed from the heat. First the crème freche and then the whipping cream was added to the juices in the frying pan. This mixture was mixed thoroughly, was not boiled. Salt was added if necessary. The fish was then served with the mixture.

This meal contained 484 calories, 5.5 g carbohydrates, 56.7 g fat, and 22.4 g protein. The ratio was 3.2:1.

Day 4.

The total calories for day 4 were 1611 calories. The total carbohydrates for day 4 were 11.3 g carbohydrates. The total fat for day 4 was 164.1 g fat. The total protein for day 4 was 42.2 g protein. The total ratio for day 4 was 3.06:1.

Breakfast—

Omelette with spinach

Ingredients:

One egg

Butter 20 g

Spinach 50 g

Whipping cream 2 tbsp

Pepper ⅛ tsp

Method:

Steamed spinach was chopped into small pieces. The egg was mixed with cream, pepper and salt in a bowl. The butter was melted in a frying pan. The spinach and egg mixture were added to the frying pan and fried.

This meal contained 301 calories, 0.6 g carbohydrates, 34.8 g fat, and 8.5 g protein. The ratio was 3.8:1.

Additionally, with the meal, fruit tea and 1 sachet splenda provided 3 calories and 1 g carbohydrates.

Am Snack—

Celery soup

Ingredients

Celery 100 g

Butter 15 g

Bouillon ¼ cube

Pepper black ⅛ tsp

Method:

Finely chopped celery was simmered in butter until soft (15-20 minutes). About 100 ml of water and the chicken cube was added and the resulting mixture was boiled for 10 min. The mixture was liquidized, mixed with pepper and salt if necessary and made up to 150 ml with water.

This meal contained 118 calories, 2.4 g carbohydrates, 12.4 g fat, and 1 g protein. The ratio was 3.7:1.

Lunch—

Salmon salad

Ingredients:

Salmon (red, tined in brine) 50 g

Mayonnaise 30 g

Whipping cream 2 tbsp

Watercress 30 g

Cayenne pepper ⅛ tsp

Method:

The salmon was broken into small pieces. Mayonnaise and whipped cream were added, and the resulting mixture was thoroughly mixed. Cayenne pepper was added and this mixture was mixed again.

This meal contained 392 calories, 0.4 g carbohydrates, 40.6 g fat, and 13.7 g protein. The ratio was 3.1:1.

Pm Snack—

Raspberry shake

Ingredients:

Raspberries 50 g

Half and half 2 tbsp

Whipping cream 2 tbsp

Splenda 1 sachet

Method:

The raspberries were liquidized in 80 ml of water. The half and half, splenda and whipped cream were added to the liquidized mixture. The mixture was made up to 150 ml with water.

The meal contained 200 calories, 3.2 g carbohydrates, 18.3 g fat, and 2.6 g protein. The ratio was 3.1:1.

Dinner—

Pork chops and mushroom sauce

Ingredients:

Pork chops 60 g

Onion 20 g

Portabella mushrooms 50 g

Oil 2 tbsp

Sour cream 50 g

Whipping cream 2 tbsp

Method:

Thin slices of onion were sautéed in oil on low heat for 5 minutes. Thinly cut mushrooms were added and this was cooked for another 5 min. The heat was increased to high and the chops sprinkled with salt and fried on each side for about 5 minutes. The chops were placed on a warm plate and the heat was switched off. The creams were added to the juices in the frying pan and salt was added if necessary. The sauce was poured over the meat.

This meal contained 599 calories, 3.7 g carbohydrates, 58.0 g fat, and 16.4 g protein. The ratio was 2.9:1.

Day 5.

The total calories for day 5 were 1578 calories. The total carbohydrates for day 5 were 20.5 g carbohydrates. The total fat for day 5 was 166.7 g fat. The total protein for day 5 was 30.5 g protein. The total ratio for day 5 was 3.3:1.

Breakfast—

Mascarpone with blueberries

Ingredients:

Blueberries 50 g

Mascarpone 60 g

Whipping cream 1½ tbsp

Splenda 1 sachet

Method:

The cream was whipped and mixed with the other ingredients.

The meal contained 362 calories, 6.0 g carbohydrates, 24.3 g fat, and 0.4 g protein. The ratio was 3.8:1.

Am Snack—

Spinach shake

Ingredients:

Spinach 80 g

Crème freche 50 g

Whipping cream ½ tbsp

Method:

The spinach was steamed and about 100 ml of the fluid was kept. This was liquidized and mixed with the cream, and made up to 150 ml with water. Salt was added to taste.

The meal contained 150 calories, 2.1 g carbohydrates, 22.8 g fat, and 4.1 g protein. The ratio was 3.1:1.

Lunch—

Cauliflower with egg

Ingredients:

Cauliflower 70 g

Butter 30 g

Pesto 28 g

One egg

Method:

The cauliflower was broken into small florets and boiled in salted water until soft (about 10 min). The cauliflower was drained. The cauliflower was broken into very small pieces using a fork. The butter was melted in a frying pan. Mashed cauliflower, pesto and the egg were added to the frying pan. Everything was mixed together and fried until the egg was done. More salt was added if needed.

This meal contained 394 calories, 3.5 g carbohydrates, 43.0 g fat, and 9.9 g protein. The ratio was 3.2:1,

Pm Snack—

Zucchini soup

Ingredients:

Zucchini 80 g

Chicken bouillon ¼ cube

Butter 15 g

Method:

Thin slices of zucchini were sautéed in butter until soft. 100 ml of boiling water with bouillon cube dissolved in it was added and boiled for 30 sec. The resulting mixture was made up to 150 ml with water.

This meal contained 147 calories, 3.1 g carbohydrates, 12.9 g fat, and 1.3 g protein. The ratio was 3.0:1.

Additionally, with this meal, fruit tea and 1 sachet splenda provided 3 calories and 1 g carbohydrates.

Dinner—

Beef with dill sauce

Ingredients:

Beef brisket 60 g

Butter 30 g

Dill 30 g

Crème freche 50 g

Whipping cream 1 tbsp

Cauliflower 50 g

Method:

The meat was boiled. The cauliflower was boiled in salted water until soft. The cauliflower was drained and crushed using a fork. Finely chopped dill was fried in melted butter. 1 tbsp of the fluid saved from when the beef was boiled, crème freche, crushed cauliflower and meat cut into thin slices was added to the dill. Salt was added to taste.

This meal contained 522 calories, 4.8 g carbohydrates, 63.7 g fat, and 14.8 g protein. The ratio was 3.2:1.

Day 6.

The total calories for day 6 were 1578 calories. The total carbohydrates for day 6 were 19.9 g carbohydrates. The total fat for day 6 was 166.7 g fat. The total protein for day 6 was 28.6 g protein. The total ratio for day 6 was 3.44:1.

Breakfast—

Omelette with spring onion

Ingredients:

One egg

Oil 2 tbsp

Spring onion 40 g

Pepper ¼ tsp

Method:

Finely chopped spring onion was fried in the oil until very soft. Salt and pepper were added. The egg was whisked and made into an omelet.

This meal contained 324 calories, 4.0 g carbohydrate, 32.2 g fat, and 7.2 g protein. The ratio was 2.9:1.

Am Snack—

Strawberry shake

Ingredients:

Strawberries 70 g

Whipping cream 3 tbsp

Splenda 1 sachet

Method:

The strawberries were liquidized in 80 ml water. Splenda and whipped cream were added and the resulting mixture was made up to 150 ml with water.

This meal contained 175 calories, 4.4 g carbohydrates, 18.2 g fat, and 0.6 g protein. The ratio was 3.6:1.

Lunch—

Avocado cream and Chinese cabbage

Ingredients:

Avocado 70 g

Mayonnaise 40 g

Black pepper ¼ tsp

Dill fresh 5 g

Chinese cabbage 70 g

Method:

The avocado was scooped from the shell and cut into small pieces. Mayonnaise, pepper and finely chopped dill were added and the resulting mixture was made into a smooth paste using a hand blender. Salt was added to taste. The paste was spread onto individual leaves of Chinese cabbage.

This meal contained 401 calories, 7.0 g carbohydrates, 42.6 g fat, and 2.7 g protein. The ratio was 3.6:1.

Pm Snack—

Broccoli soup

Ingredients:

Broccoli 40 g

Butter 20 g

Chicken bouillon ¼ cube

Whipping cream ½ tbsp

Method:

Small florets of broccoli were covered with water in a saucepan and the bouillon cube was added to the broccoli and water. The broccoli was boiled until soft (about 5 minutes). The butter was added, and once the butter was melted, the resulting mixture was liquidized. The cream was added to the mixture and then the mixture was made up to 150 ml with water.

This meal contained 158 calories, 2.2 g carbohydrates, 19.5 g fat, and 3.3 g protein. The ratio was 3.6:1.

Additionally, with this meal, fruit tea and 1 sachet splenda provided 3 calories and 1 g carbohydrates.

Dinner—

Beef with watercress

Ingredients:

Beef brisket 80 g

Butter 30 g

Watercress 150 g

Whipping cream 2 tbsp

Horseradish sauce 10 g

Method:

The meat was boiled and then put aside. The watercress was sautéed in butter until it wilted. The whipping cream, horse radish sauce and 2 tbsp of the fluid in which the meat had been boiled were added to the watercress. The meat and salt, if needed, were then added to the watercress.

The meal contained 519 calories, 1.3 g carbohydrates, 56.0 g fat, and 14.8 g protein. The ratio was 3.5:1.

Day 7.

The total calories for day 7 were 1599 calories. The total carbohydrates for day 7 were 19.9 g carbohydrates. The total fat for day 7 was 193.1 g fat. The total protein for day 7 was 35.7 g protein. The total ratio for day 7 was 3.47:1.

Breakfast—

Omelette with asparagus

Ingredients:

One egg

Butter 20 g

Asparagus 50 g

Whipping cream 2 tbsp

Pepper ⅛ tsp

Method:

Pieces of asparagus about ½ inch long were boiled in about 100 ml salted water until they are soft (about 10 minutes). The asparagus was drained. The egg, whipping cream and pepper were mixed in a bowl. The asparagus was added to this mixture. The butter was melted in a frying pan and the mixture was poured into the frying pan to make an omelet. Salt was added to taste if needed.

This meal contained 299 calories, 1.2 g carbohydrates, 34.7 g fat, and 7.9 g protein. The ratio was 3.8:1.

Am Snack—

Bouillon with watercress

Ingredients:

Chicken bouillon ¼ cube

One egg yolk

Butter 10 g

Watercress 2 sprigs

Whipping cream 1 tbsp

Method:

The bouillon was dissolved in about 80 ml of boiling water in a saucepan. The butter and egg yolk were whisked in and the resulting mixture was boiled for about 1 minute. The chopped watercress was added to the mixture and the mixture was removed from the heat. The cream was added to the mixture and the mixture was made up to 150 ml with warm water.

This meal contained 166 calories, 1.2 g carbohydrates, 18.4 g fat, and 4.1 g protein. The ratio was 3.4:1.

Lunch—

Eggplant and red pepper

Ingredients:

Eggplant 70 g

Red pepper 70 g

Oregano 1 tsp

Mayonnaise 40 g

Garlic powder 1 tsp

Whipping cream 3 tbsp

Iceberg lettuce 50 g

Method:

The eggplant (about 100 g) and pepper were baked at 350° F. for 20 minutes. The eggplant was peeled and 70 g of each was used. The eggplant was cut into pieces. Oregano, garlic powder, and salt to taste were added to the eggplant and the resulting mixture was liquidized. Mayonnaise and whipped cream were added to the mixture and the mixture was mixed into a smooth paste. This paste was mixed with chopped Chinese cabbage leaves.

This meal contained 486 calories, 10.4 g carbohydrate, 50.4 g fat, and 2.9 g protein. The ratio was 3.8:1.

Pm Snack—

Asparagus shake

Ingredients:

Asparagus 70 g

Crème freche 40 g

Whipping cream 1 tbsp

Pepper black ⅛ tsp

Chili powder ⅛ tsp

Water from boiled asparagus

Method:

The asparagus was boiled in about 800 ml of salted water until soft. The asparagus was liquidized in the fluid in which it was boiled. The creams, pepper, chili powder and salt, if necessary, were added to the liquidized mixture. This mixture was mixed and made up to 150 ml with water.

This meal contained 151 calories, 2.6 g carbohydrates, 27.8 g fat, and 3.0 g protein. The ratio was 4.6:1.

Additionally, with this meal, fruit tea and 1 sachet splenda provided 3 calories and 1 g carbohydrates.

Dinner—

Salmon with spinach

Ingredients:

Salmon (Atlantic, farmed) 70 g

Lemon juice 10 g

Lemon pepper ¼ tsp

Butter 20 g

Crème freche 50 g

Whipping cream 3 tbsp

Spinach 100 g

Method:

The fish was sprinkled with lemon juice, lemon pepper and salt. The butter was melted on medium heat and the fish was fried for 3-4 minutes on each side. The spinach was steamed until it wilted. The spinach was then put in a bowl and mixed with the creams and the juice from the fish. Salt was added to taste if necessary. This spinach mixture was served with the fish.

This meal contained 494 calories, 3.5 g carbohydrate, 61.8 g fat, and 17.8 g protein. The ratio was 2.9:1.

Day 8.

The total calories for day 8 were 1623 calories. The total carbohydrates for day 8 were 17.4 g carbohydrates. The total fat for day 8 was 182.2 g fat. The total protein for day 8 was 39.6 g protein. The ratio was 3.2:1.

Breakfast—

Omelette with tomato and Chinese cabbage

Ingredients:

One egg

Butter 20 g

Tomato 100 g

Chinese cabbage 50 g

Oil 1 tbsp

Method:

Small pieces of the Chinese cabbage were fried in oil for 1 minute. Butter and small pieces of peeled tomato were added and fried for 2 more minutes. The egg was whisked in, salt was added, and an omelet was made.

This meal contained 333 calories, 3.6 g carbohydrates, 36.3 g fat, and 8.0 g protein. The ratio was 3.1:1.

Am Snack—

Celery shake

Ingredients:

Celery 100 g

Crème freche 50 g

Pepper black ⅛ tsp

Method:

Pieces of celery about ½ inch long were boiled in about 80 ml of salted water. The celery was liquidized and mixed with pepper and crème freche. The resulting mixture was made up to 150 ml with water.

This meal contained 124 calories, 3.6 g carbohydrates, 19.7 g fat, and 2.5 g protein. The ratio was 3.2:1.

Lunch—

Sardines

Ingredients:

Sardines 50 g

Pickled cucumbers 100 g

Mustard powder ¼ tsp

Pepper ⅛ tsp

Oil from tin 3 tbsp

Method:

The sardines were broken into small pieces and mixed with coarsely grated pickled cucumbers, mustard, pepper and oil. Salt was added to taste if needed.

This meal contained 473 calories, 0.9 g carbohydrate, 46.1 g fat, and 12.7 g protein. The ratio was 3.4:1.

Pm Snack—

Spinach soup

Ingredients:

Spinach 50 g

Crème freche 30 g

Method:

The spinach was steamed until it wilted and then was chopped. The fluid was kept. The crème freche and salt, if needed, if needed was added to the spinach. The resulting mixture was liquidized and made up to 150 ml with the fluid left from steaming the spinach.

This meal contained 76 calories, 1.3 g carbohydrates, 11.9 g fat, and 2.5 g protein. The ratio was 3.1:1.

Additionally, with this meal, fruit tea and 1 sachet splenda provided 3 calories and 1 g carbohydrates.

Dinner—

Szeged goulash (Hungarian style goulash with cabbage)

Ingredients:

Pork shoulder 60 g

Onion 30 g

Paprika 1 tsp

White cabbage 50 g

Sauerkraut 50 g

Oil 3 tbsp

Crème freche 50 g

Method:

The chopped onion was sautéed in oil for 2-3 min with paprika and salt. The heat was increased and small pieces of meat were added, which were fried until brown with constant stirring. The heat was reduced. Strips of white cabbage were added and cooked until the meat was tender (about 30-40 minutes). The sauerkraut was added and this mixture was cooked for another 5 minutes. The mixture was removed from the heat and the crème freche and more salt, if necessary, were added to the mixture. The mixture was mixed.

This meal contained 614 calories, 7.0 g carbohydrate, 68.2 g fat, and 13.9 g protein. The ratio was 3.3:1.

### Example 3

### Treatment of Type 2 Diabetes

A study was conducted to examine the effects of a ketogenic diet on subjects having obesity-related type 2 diabetes. The study treated 14 obese subjects having type 2 diabetes, treated either with the ketogenic diet (i.e., received the above-described ketogenic food composition; n=7) or with weight loss medication (i.e., orlistat) and dietary counseling (n=4) or with counseling alone (n=3). All 14 obese subjects had a BMI greater than or equal to 30 kg/mg² before initiation of the study.

Orlistat 360 mg/day treatment for one year reduces mean fasting plasma glucose (FPG) by −25 mg/dl and HbA1c by −0.74% (vs. 8.46 mg/dl and −0.31%, respectively, for PBO). See, for example, http://www.novonordisk.com/include/asp/exe_news_attachmentasp?sAttachmentGUID=3f254ec7-1b91-4334-bf0d-572d4e6a4c3e, accessed 8 Feb. 2015. 24% patients discontinue or decrease the dosage of at least one diabetic medication, compared with 18% of PBO, while 12% of orlistat patients increase medication load compared with 22% of PBO group. See, for example, Jacob S, Rabbia M, Meier M K, Hauptman J. Orlistat 120 mg improves glycemic control in type 2 diabetic patients with or without concurrent weight loss. Diabetes Obes Metab. 2009 April; 11(4):361-71. 6% of insulin-treated type 2 diabetes patients stopped insulin vs. none in PBO. See, for example, Rowe R, Cow M, Poole C, McEwan P, Morgan C, Walker M. The effects of orlistat in patients with diabetes: improvement in glycemic control and weight loss. Curr Med Res Opin. 2005 November; 21(11):1885-90.

All subjects were taking medication to treat the type 2 diabetes prior to initiation of the study. Specifically, 5 of the obese subjects having type 2 diabetes and treated with the ketogenic diet were taking 1 to 2 oral hypoglycemic medications and 2 of the obese subjects having type 2 diabetes were taking insulin and oral medications. All subjects treated with the ketogenic diet discontinued diabetic medications within 2 months of starting the ketogenic diet. All subjects not receiving the ketogenic diet remained on diabetic medications.

The subjects were treated with the above-described ketogenic food composition from about 2 months to about 6 months as a sole diet or source of calories. Specifically, the ketogenic food composition had a ratio of 3:1. All subjects receiving the ketogenic food composition (i.e., the ketogenic diet) tolerated the ketogenic diet well. None of the subjects receiving the ketogenic diet stopped the diet due to side effects.

Prior to initiation of the study, the blood glucose level, insulin level, and glycosylated hemoglobin (HbA1C) level were measured for each subject to establish a respective baseline for these levels. For each subject, the blood glucose level, insulin level, and HbA1C level was measured during the course of the study and at the conclusion of the study. The blood glucose levels, insulin levels, and HbA1C levels were measured by techniques known to those skilled in the art.

At 6 months into the study, the seven obese subjects with type 2 diabetes and receiving the ketogenic diet had discontinued all diabetic medications and had improved blood glucose levels, insulin levels, and glycosylated hemoglobin (HbA1C). All subjects not receiving the ketogenic diet remained on diabetic medications.

Additionally, all seven of the obese subjects having type 2 diabetes and receiving the ketogenic diet had lost weight as measured by body mass index (BMI). Specifically, all seven subjects had lost greater than 5% of BMI relative to their respective BMI prior to initiation of the study. Four of seven subjects had lost greater than or equal to 10% of BMI relative to their respective BMI prior to initiation of the study. All subjects receiving weight loss medication and dietary counseling and all subjects receiving dietary counseling alone had lost 5% BMI relative to their respective BMI prior to initiation of the study. None of the subjects receiving weight loss medication and dietary counseling had lost greater than or equal to 10% BMI relative to their respective BMI prior to initiation of the study. None of the subject receiving dietary counseling alone had lost greater than or equal to 10% BMI relative to their respective BMI prior to initiation of the study.

In summary, this study demonstrated that treatment of obese subjects having type 2 diabetes with the ketogenic diet resulted in improvements in blood glucose level, insulin level, and HbA1C such that all diabetic medications were no longer needed and thus, discontinued. Accordingly, the ketogenic food composition described herein, when having the ratio of 3:1, was effective in treating obesity-related type 2 diabetes.

### Example 4

### Additional Treatment Study of Type 2 Diabetes

A study is conducted to compare the effects of the following three treatments on obese subjects having type 2 diabetes: (1) treatment with the ketogenic diet using the ketogenic food composition described herein; (2) weight loss treatment with a standard diet; and (3) weight loss medication. 150 subjects are enrolled in the study, including normal, healthy subjects, obese subjects without type 2 diabetes, and obese subjects having type 2 diabetes.

Prior to initiation of the study, the blood glucose level, insulin level, and glycosylated hemoglobin (HbA1C) level are measured for each subject to establish a respective baseline for these levels. For each subject, the blood glucose level, insulin level, and HbA1C level are measured during the course of the study and at the conclusion of the study. The blood glucose levels, insulin levels, and HbA1C levels are measured by techniques known to those skilled in the art.

During the course of the study, the sole diet of the subjects is the ketogenic food composition described herein. Specifically, the ratio of the ketogenic food composition is 3:1.

At the conclusion of the study, treatment with the ketogenic food composition described herein has facilitated weight loss in the obese subjects having type 2 diabetes as demonstrated by BMI measurements. The treatment with the ketogenic food composition described herein facilitated a reversal of insulin resistance and hyperinsulinemia in the obese subjects having type 2 diabetes as demonstrated by measurement of blood glucose level, insulin level, and HbA1C. These improvements in weight, insulin resistance, and hyperinsulinemia are maintained after treatment with the food composition described herein has ended. Accordingly, this study demonstrates the efficacy of the ketogenic food composition described herein in the treatment of type 2 diabetes in obese subjects.

### Example 5

### Treatment of Obstructive Sleep Apnea

A study was conducted to examine the effects of the ketogenic diet on subjects having obstructive sleep apnea. The study included five obese subjects with obstructive sleep apnea. All subjects had a BMI of greater than or equal to 30 kg/m².

All five of the obese subjects with obstructive sleep apnea were undergoing treatment prior to initiation of the study, namely continuous positive air pressure (CPAP). During the course of the study, the subjects were treated with the above-described ketogenic food composition as a sole diet or source of calories. Specifically, the ketogenic food composition had a ratio of 3:1.

Prior to initiation of the study, the apnea/hypopnea index (AHI), which was a polysomnographic measure of the severity of obstructive sleep apnea, was determined for each subject. By the conclusion of the study, the AHI of all five of the obese subjects with obstructive sleep apnea had improved by greater than 50% relative to the AHI measured prior to initiation of treatment with the food composition described herein. In 3 of the 5 obese subjects with obstructive sleep apnea, sleep apnea had remitted and these 3 subjects were able to discontinue CPAP treatment. In 2 of the 5 subjects, the AHI value returned to normal (i.e., an AHI value of less than or equal to 5).

In summary, this study demonstrated that treatment of obese subjects with obstructive sleep apnea resulted in improved AHI, i.e., less severe obstructive sleep apnea, and remittance of sleep apnea. Accordingly, the ketogenic food composition described herein, when having the ratio of 3:1, was effective in treating obesity-related obstructive sleep apnea.

### Example 6

### Additional Treatment Study of Obstructive Sleep Apnea

A study is conducted to compare the effects of the following three treatments on obese subjects with obstructive sleep apnea: (1) treatment with the ketogenic diet using the ketogenic food composition described herein; (2) weight loss treatment with a standard diet; and (3) weight loss medication. 150 subjects are enrolled in the study, including obese subjects with obstructive sleep apnea.

Prior to initiation of the study, the apnea/hypopnea index (AHI) was determined for each subject. During the course of the study, the sole diet of the subjects is the ketogenic food composition described herein. Specifically, the ratio of the ketogenic food composition is 3:1.

At the conclusion of the study, treatment with the ketogenic food composition described herein facilitated in the obese subjects with obstructive sleep apnea an improvement in AHI relative to the AHI measured prior to initiation of the treatment with the ketogenic food composition described herein. It is expected that a significant number of the obese subjects with obstructive sleep apnea show remittance of sleep apnea and no need for CPAP treatment. Accordingly, this study demonstrates the efficacy of the ketogenic food composition described herein in the treatment of obstructive sleep apnea.

### Example 7

### Treatment of Glioblastoma Multiforme

A study was conducted to examine the effects of the ketogenic diet on a subject having advanced glioblastoma multiforme (GBM) that had failed to respond to standard treatments for GBM. Standard treatments for GBM included radiation therapy and chemotherapy using temozolamide. For such a subject, the average survival was 3 months.

During the course of the study, the subject was treated for 6 months with the above-described ketogenic food composition as a sole diet or source of calories in combination with avastin. Specifically, the ketogenic food composition had a ratio of 4:1. The subject also received avastin during the course of the study.

By the conclusion of the study, the symptoms of GBM in the subject had almost cleared and the size of the tumor had shrunk as measured by magnetic resonance imaging (MRI) as compared to the size of the tumor prior to initiation of the treatment with the ketogenic food composition. 12 months after initiation of the treatment with the ketogenic food composition, the subject was doing well.

In summary, this study demonstrated that treatment with the ketogenic food composition described herein, as an adjuvant to another cancer therapy, resulted in the shrinkage of the tumor and improvement in the symptoms associated with GBM. Accordingly, the ketogenic food composition described herein, when having the ratio of 4:1, was effective in treating GBM.

### Example 8

### Additional Treatment Study of Glioblastoma Multiforme

A study is conducted examining the treatment of subjects with newly diagnosed GBM with the ketogenic food composition described herein as an adjuvant therapy to radiation therapy and chemotherapy. During the course of the study, the sole diet of the subjects is the food composition described herein. Specifically, the ratio of the ketogenic food composition is 4:1.

At the conclusion of the study, it is expected that treatment with the ketogenic food composition described herein acts as an effective adjuvant to radiation therapy and chemotherapy, aiding the reduction of tumor size and symptoms associated with GBM. Accordingly, this study demonstrates the efficacy of the ketogenic food composition herein as an adjuvant therapy in the treatment of glioblastoma multiforme.

### Example 9

### Evaluation of a 5-Week Meal Cycle

The subjects enrolled in the pilot studies described above in Examples 3, 5, and 7 evaluated the palatability, appearance, and satiety of the ketogenic food composition. The scale for evaluation was 0 to 5, in which 0 was terrible and 5 was excellent.

Specifically, the ketogenic food composition was a recurrent cycle of 5 weeks, in which five ketogenic food compositions (i.e., meals) were ingested per day. As such, a total of 175 food compositions were evaluated by the subjects (i.e., 5 weeks×7 days/week×5 meals/day).

The results showed that 80% of the 175 ketogenic food compositions were evaluated as 4 or greater with regards to palatability and appearance. The results also showed that greater than 90% of the 175 ketogenic food compositions were evaluated as 5 or greater with regards to satiety. Accordingly, these results demonstrated that the palatability of, appearance of, and satiety provided by the ketogenic food composition was well-received by the subjects undergoing treatment, which in turn, facilitated adherence to the treatment regimen.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### Example 10

### Ketogenic Diet for Patients with Epilepsy

Adults with refractory epilepsy treated adjunctively with 3:1 KD with 1600 kcal/day restriction. 12 subjects were treated. 1 subject dropped out after 4 days because of psychosocial reasons. Of the remaining 11 subjects, 8 subjects were obese, 1 was overweight and 2 had normal weight. Treatment lasted 4 months initially, followed by an elective open-ended extension, entered by 10/11 subjects, with treatment duration range of 6-26 months. 1 other subject stopped the diet for social reason after 1.5 months. All 11 subjects lost weight. Mean BMI declined by 18.3% from 33.8 kg/m² to 27.5 kg/m² at study end. Among the 9 obese (n=8) and overweight (n=1) subjects, mean BMI declined by 20.1% from 36.07 kg/m² to 28.8 kg/m², 89% of patients had ≧10% BMI reduction, 78%≧15%, and 44%≧25% (Table 1). Weight loss stabilized after 8-12 months.

Table 1. Weight, BMI and % BMI reduction in 9 adults with intractable epilepsy and co-morbid obesity (n=8) or overweight (n=1) treated with 3:1 ketogenic diet. Treatment lasted for more than 6 months in 8 subjects, 1.5 months in 1 subject.

TABLE 1

| | Mean | Mean | ≧10% | | ≧25% | ≧30% |
|---|---|---|---|---|---|---|
| No. of | weight | BMI | BMI | ≧15% BMI | BMI | BMI |
| Subjects | (lb) | (kg/m²) | (kg/m²) | (kg/m²) | (kg/m²) | (kg/m²) |
| 9 | 65.4 | 20.4 | 89% | 78% | 44% | 22% |

The results presented in Table 1 are unlike any other available weight loss treatments except for bariatric surgery. Side effects, all mild, included nausea (n=2), diarrhea (n=2), constipation (n=2) and mild transient hunger (n=3, lasting ≦1 weeks). No subject stopped treatment because of side effects. Mean cholesterol increased from 213.7 to 257.8 md/dl (without other significant lipid changes), with return to pre-study baselines in subjects with cholesterol elevation within 3 months of ketogenic diet discontinuation.

Mean fasting glucose levels declined from 87.7 mg/dl baseline to 78.7 mg/dl. One patient with co-morbid NIDDM and overweight normalized fasting plasma glucose level on ketogenic diet treatment day 3, while stopping one of two oral hypoglycemics. Fasting insulin levels declined from 16.4 μIU/ml to 2.8 μIU/ml on ketogenic diet day 4. FPG levels remained low normal during the 6 months of treatment. HbA₁C level dropped from 7 at baseline to 5.2 at ketogenic diet month 6. The patient's BMI declined from 28.9 kg/m² to 24 kg/m².

### Example 11

Evaluation of a 3:1 ketogenic diet treatment in patients with obesity and type 2 diabetes. 17 patients were treated in 3 open label parallel treatment arms of ketogenic diet (n=10), Orlistat 360 mg/day and lifestyle/dietary counseling (n=4) and lifestyle counseling only (n=3). The treatment lasted 9 months. The first 5 subjects were enrolled into the ketogenic diet arm (“front-loaded”). After that, treatment assignment was randomized. Ketogenic diet treatment included a 3:1 [fat]: [protein+carbohydrate] by weight diet, with daily 20 g carbohydrates and a 1600 kcal restriction. Ketogenic diet food included an all-inclusive ready-made meal plan. Meals were made according to the recipes described herein. Five meals a day were given to the patients. The meals were delivered frozen to a central site once for patients to pick up. Patients ate no other food, and drank only 0 calorie-drinks. Lifestyle counseling was based on the LEARN (Lifestyle, Exercise, Attitudes, Relationships, and Nutrition. Patients in all 3 arms were instructed to exercise ≧30 minutes/day. Patients checked their blood glucose at least 2×/day, morning fasting and evening 2 hours post-prandial. They checked urine for ketone levels using urine ketostix (Bayer AG, Germany), and kept a daily exercise log.

Because of concern for hypoglycemia, patients on ketogenic diet had oral hypoglycemic medication reduced by 50% on treatment day one, and then adjusted as clinically mandated, based on their glucose diaries and laboratory-measured FPG levels. Patients on oral hypoglycemics and insulin had initial adjustment of oral hypoglycemic only, with initial 3×/day monitoring of blood glucose, and insulin adjustment based on glucose levels. Patients in the other 2 arms had hypoglycemic medications adjusted based on their blood glucose and levels. Outcome measures included weight change, fasting plasma glucose and insulin levels, HbA1C, medication adjustment, and fasting lipid levels.

All 17 subjects received at least one treatment (“intent to treat”, ITT): 9 in the ketogenic diet group, 4 in Orlistat+counseling, and 3 in counseling alone. 10 patients discontinued the study early: 5/10 in ketogenic diet, 2/2 in Orlistat and 2/3 in counseling groups. Discontinuations occurred at ≦3 weeks (n=4, 1 ketogenic diet, 1 counseling, 2 orlistat,), 2 months (n=2, both ketogenic diet), 4 months (n=2, 1 each in ketogenic diet and counseling) and 8 months (n=1, ketogenic diet). Reasons for discontinuation were psychological (n=2, 1 ketogenic diet, 1 counseling, due to anxiety about dietary restriction with pre-study anxiety and depression), loss of motivation (n=2, 1 ketogenic diet, 1 counseling), socioeconomic (n=3, all ketogenic diet, due to inability to pick up food because of a long drive or work-related travel), and adverse events (n=2, both Orlistat, diarrhea+/− abdominal cramps). Thus 7 subjects completed full 9 months of treatment, 4/9 ketogenic diet, 2/4 Orlistat and 1/3 counseling subjects. All but one of the subjects who discontinued treatment early was evaluated at 9 months. Results were evaluated both as “intent to treat” (ITT, n=17:10/4/3) and “per protocol” (PP), i.e., patients who completed the full 9 months of treatment (N=7:4/2/1).

Results are shown in Table 2. For the 5 subjects who completed the full 9 months of treatment (“per protocol”, PP), the ketogenic diet group had mean BMI 17.1%% (from 37.4 to 31.06 kg/m²). Weight loss plateaued at 8 months.

All subjects achieved T2D remission. Mean FPG declined by 47 mg/dl (from 135.8 to 88.8 mg/dl), fasting insulin by 15.9 IU/ml (from 20.56 to 4.64 mIU/dl) and HbA1C by 1.36% (from 7 to 5.64%). All subjects discontinued all hypoglycemic medications. (Baseline medications were: metformin 2000 mg daily, metformin 2550 mg, metformin 2000+actos 15 mg, and Actoplus with metformin/acto 1000/15 mg). In all diabetic medications were halved on day 2 of the diet. They were stopped completely by 2 weeks of the diet in 3/4, and by 1 month on the diet in 1 subject.

One of the 5 PP ketogenic diet subject 1 has continued to be off diabetic, antihypertensive and hyperlipidemic medications for 1 year after diet discontinuation, with stable weight. The other 4 have only had 6 months of follow up. All 3 have remained off diabetic medications with normal glucose and HbA1 levels.

In the non-ketogenic diet (NKD) PP groups combined (Orlistat and counseling and counseling alone), mean BMI declined by 8%. There were no type 2 diabetes remissions. FPG fell by 2.4 mg/dl (from 137 to 134.7 mg/dl), mean fasting insulin level by 3.5 IU/ml (from 16.3 to 12.8 IU/ml) and HbA1C by 1% (from 7.4% to 6.4%). Medications did not change.

Results for the whole intent to treat population are also shown in Table 2. In all ketogenic diet ITT subjects, mean BMI declined by 10.8% (from 40.3 to 35.9), FPG by 25.5 mg/dl (from 143.1 to 117.6 mg/dl), insulin by 6.9 IU/ml (from 20.06 to 13.14 IU/ml), and HBA1C by 1.2% (from 7.5% to 6.3). All 9 subjects had all medication discontinued while on the diet, 7 by 1 month of treatment and 1 by 2 months of treatment. This included 2 subjects on insulin, 1 each on 80 and 115 units/day, who stopped insulin 1 and 2 months after ketogenic diet initiation. Both subjects stopped the diet after 2 months because of logistic difficulties in picking up the food (too long a drive and work-related travel). They both resumed insulin.

In the non-ketogenic (non-ketogenic diet) ITT patients, mean BMI declined by 4.6% (from 36.6 to 34.9 kg/m²), FPG by 4.3 mg/dl (from 131.3 to 127 mg/dl), insulin by 7.4 IU/ml (from 19.9 to 12.6 IU/ml), and HBA1C by 0.5% (from 7.2% to 6.7%). No subjects changed diabetic medication (all patients were on oral hypoglycemic medication, none on insulin). At study end. 5/10 ITT subjects (50%) were in remission, off diabetic medications.

Table 2: weight loss, glycemic change, and medications discontinuation in our pilot study (unpublished data) after 9 months of treatment (rounded to the nearest decimal point).

TABLE 2

| | PP | | ITT | |
|---|---|---|---|---|
| | | NKD | | NKD |
| | KD (n = 5) | (n = 3) | KD (n = 10) | (n = 7) |
| Weight loss (lb) | 35.2 | 12.3 | 25.5 | 8 |
| % BMI reduction | 17.1 | 8 | 10.8 | 4.6 |
| ↓ FPG (mg/dl) | 47 | 2.3 | 25.5 | 4.3 |
| ↓ Fasting Insulin (μIU/L) | 15.9 | 3.5 | 6.9 | 7.4 |
| ↓ HbA1C (%) | 1.36 | 1 | 1.2 | 0.5 |
| DM Medication | 100% | 0% | 50% | 0% |
| withdrawal | | | | |

PP=per protocol; ITT=intent to treat. KD=ketogenic diet. NKD=non-ketogenic diet groups combined, including orlistat and counseling group and counseling alone group.

BMI=body mass index; FPG=fasting plasma glucose, HbA1c=glycosylated hemoglobin, TG=triglycerides, HDL=high density lipoprotein.

The tempo of glycemic normalization in the ketogenic diet group was fast. Pretreatment FPG was elevated in 5 ketogenic diet ITT patients at baseline. It normalized in 3 patients by the end of first week of treatment, in 1 patient by 4 weeks, and in 1 (insulin-treated) by 6 weeks. Other patients had normal FPG before study but were able to discontinue medications by 1-2 weeks after ketogenic diet initiation.

In addition, all 4 ketogenic diet per protocol patients had hypertension. 2 of 4 were able to stop anti-hypertensives (after 1 and 2 months), with normal blood pressure. 2 of 3 of the NKD PP subjects had hypertension. None stopped the antihypertensive medications.

The ketogenic diet was well tolerated. No patient discontinued the diet because of side effects. 2 patients had transient hunger, one moderate, one mild. 1 patient developed significant hypercholesterolemia (highest cholesterol level of 369 mg/dl). There were no other side effects. 2 non-ketogenic diet patients had side effects, both in the orlistat group, diarrhea, and one patient, in addition, abdominal cramps, both resulting in early treatment discontinuation.

This data suggests that a 3:1 ketogenic diet is unexpectedly effective in inducing glycemic normalization and type 2 diabetes remission in obese individuals with type 2 diabetes, and may be more effective in weight loss than any other dietary or pharmacological approach available to-day. Thus it can be expected to be effective in type 2 diabetes prevention in obese individuals. 3:1 ketogenic diet appears to be markedly more effective in both type 2 diabetes treatment and in weight loss than standard LC diets. The mean BMI loss in our pilot studies with classic ketogenic diet ranged from 9.6-18.3% compared with 6.5-12.9% loss in LC studies. Similarly, the hypoglycemic effect was dramatically greater in our pilot study than in LC studies, with 100% type 2 diabetes remission in study completers.

1. A method for treating type 2 diabetes in a subject in need thereof, the method comprising:
(a) administering one or more ketogenic food compositions comprising fat, protein, and carbohydrate to a subject in need thereof;
(b) wherein the ratio of fat to a combination of protein and carbohydrate in the one or more ketogenic food compositions is about 2.5:1 to about 4.5:1;
(c) wherein total number of carbohydrates per day administered to a subject in need thereof is less than about 20 grams throughout the entire treatment time frame; and
(d) wherein the one or more ketogenic food compositions provides about 1300 kilocalories (kcal)/day to about 2100 kcal/day to the subject in need thereof.

2. The method of claim 1, wherein said subject in need thereof is diagnosed as having type 2 diabetes prior to treatment.

3. The method of claim 1, wherein the one or more ketogenic food compositions are the sole diet of the patient.

4. The method of claim 1, wherein the specified treatment time is about at least 2 months.

5. The method of claim 1, wherein the subject in need thereof achieves a fasting blood glucose <126 mg/dl and HbA1C ≦6.4% after a specified treatment time.

6. The method of claim 1, wherein the subject in need thereof achieves remission from type 2 diabetes after about 2 months, about 4 months, and/or about 6 months.

7. The method of claim 1, wherein the subject in need thereof wherein the subject in need thereof is not prescribed and/or does not take medication and/or pharmaceuticals to treat type 2 diabetes after about 2 months, about 4 months, and/or about 6 months.

8. The method of claim 1, wherein the one or more ketogenic food compositions is a meal.

9. The method of claim 1, wherein approximately five meals per day are administered to a subject in need thereof.

10. The method of claim 1, wherein the method of treating type 2 diabetes comprises administration of only meals and does not include administration and/or consumption of a snack or shake.

11. The method of claim 1, wherein the method of treating type 2 diabetes does not vary the maximum amount of daily carbohydrate allowance over the treatment time frame.

12. The method of claim 1, wherein the fasting blood glucose level of the subject in need thereof is decreased by at least about 5%, at least about 10%, at least about 15%, at least about 25%, at least about 40%, at least about 50%, or at least about 75% or more after about 1 month, about 2 months, about 3 months, about 6 months or about 12 months or more relative to a subject with type 2 diabetes over the same time frame and not administered each of method steps (a)-(d).

13. The method of claim 1, wherein the fasting insulin of the subject in need thereof is decreased by at least about 5%, at least about 10%, at least about 15%, at least about 25%, at least about 40%, at least about 50%, or at least about 75% or more after about 1 month, about 2 months, about 3 months, about 6 months or about 12 months or more relative to a subject with type 2 diabetes over the same time frame and not administered each of method steps (a)-(d).

14. A ketogenic food composition for promoting ketogenesis in a subject in need thereof, the composition comprising fat, protein, and carbohydrate,
wherein a ratio of fat to a combination of protein and carbohydrate is about 2.5:1 to about 4.5:1, and wherein the food composition is a sole diet for the subject.

15. The ketogenic food composition of claim 14, wherein the ratio is about 3:1 to about 4:1.

16. The ketogenic food composition of claim 14, wherein the ratio is about 3:1 or about 4:1.

17. The ketogenic food composition of claim 14, wherein the ketogenic food composition is a plurality of ketogenic food compositions, and wherein in each ketogenic food composition, the ratio is about 2.5:1 to about 4.5:1.

18. A method for treating type 2 diabetes in a subject in need thereof, the method comprising: administering the ketogenic food composition of claim 14 to the subject, wherein the disease is an obesity-related disorder.

19. The method of claim 18, wherein the method further comprises reducing at least one of insulin resistance, blood glucose level, insulin level, obesity, hyperinsulinemia, glycosylated hemoglobin level, or a combination thereof in the subject.

20. The method of claim 18, wherein the ketogenic food composition is a plurality of ketogenic food compositions, and wherein in each ketogenic food composition, the ratio is about 2.5:1 to about 4.5:1.
